(19) United States
(12) Patent Application Publication
(10) Pub. No.: US 2011/0086399 A1
(43) Pub. Date: Apr. 14, 2011

(54) RECOVERY OF STILBENOIDS

(75) Inventors: Hans Peter Smits, Holte (DK); Helga David, Kobenhavn N (DK); Jochen Forster, Kobenhavn N (DK); Bo Stenhuus, Kobenhavn N (DK)

(73) Assignee: FLUXOME SCIENCES A/S, STENLOSE (DK)

(21) Appl. No.: 12/937,508

(22) PCT Filed: Apr. 8, 2009
(86) PCT No.: PCT/EP09/54219

§ 371 Date: Dec. 30, 2010

(30) Foreign Application Priority Data
Apr. 11, 2008 (GB) 0806595.5

(51) Int. Cl. C12P 7/22

(52) U.S. Cl. 435/156

(57) ABSTRACT

A cis- or trans-stilbenoid of the general formula (1) in which each of R¹, R², R³, R⁴ and R⁵ is hydrogen or hydroxy, or a glycosylated or oligomeric form thereof, such as resveratrol or pinosylvin is produced by cultivating a microorganism such as a genetically engineered yeast to produce said stilbenoid in a culture medium in solid form, and is separated by filtration or settling.

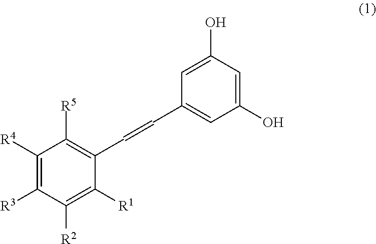

This invention relates generally to a bioreactor process in which a stilbenoid (i.e. a hydroxystilbene) is produced and recovered in solid form from the cultivation medium.

There have recently been proposed recombinant micro-organisms that have the capacity to produce certain stilbenoids of the general formula 1:

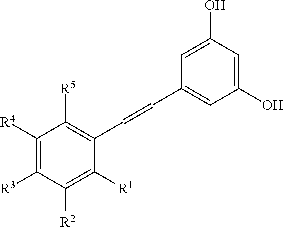

wherein each of R¹, R², R³, R⁴ and R⁵ is hydrogen or hydroxy. Examples of such compounds include resveratrol (only R³ being hydroxy), pinosylvin (all of the R groups being hydrogen) and piceatannol (R³ and R² or R⁴ being hydroxy).

One problem in the production of stilbenoid in this way lies in recovering the stilbenoid from the cultivation.

We have developed strains of yeast (and the same can be done for other fungi or of bacteria) in which the concentration of hydroxystilbene secreted into the medium by the micro-organisms is so high as to reach saturation, leading to precipitation of the product.

Moreover, we have surprisingly discovered that the precipitated stilbenoid is at least in part present as crystals sufficiently large to allow at least partial mechanical separation thereof from the cells of the micro-organism. At least in some cases, separate crystals of one stilbenoid can be at least partially separated from those of another stilbenoid on the basis of differences in size.

Accordingly, there is now provided a method for the production of a cis- or trans-stilbenoid of the general formula 1:

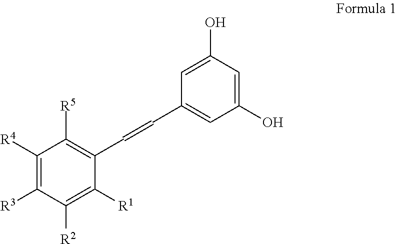

in which each of R¹, R², R³, R⁴ and R⁵ is hydrogen or hydroxy, or a glycosylated or oligomeric form thereof, comprising cultivating a micro-organism producing said stilbenoid in a culture medium, wherein said cultivation is performed so as to accumulate on or more said stilbenoids in said culture medium in solid form, and separating at least one said solid stilbenoid from the culture medium.

Preferably, the solid stilbenoids are crystalline.

Preferably, said solid stilbenoid is separated from said culture medium by filtration, by settling, decanting, or other mechanical separation methods such as flocculating and removing the micro-organisms, e.g. yeast.

As discussed below, we have observed that the stilbenoids tend to form crystals having an average particle size which is significantly larger than the size of the cells of the micro-organisms, in particular larger than yeast cells. Accordingly, the invention includes such a method in which at least one said stilbenoid has an average particle size larger than cells of said micro-organism and is at least partially separated from said micro-organism cells present in said culture medium by separating solids in said culture medium according to their size. The average particle size of said stilbenoid may be at least double the average size of said cells, for instance at least 5 times said size.

The term ‘average size’ as used in this specification is not critically dependent on the method used to determine said average. It is recognised that where there is a distribution of sizes, different average sizes may be determined by different measurement techniques such as light scattering, microscopic counting, Coulter counter and so forth.

Similarly averages for particle size distributions may be calculated in various ways, for instance as number average particle size or as weight average particle size. Generally, for making comparisons, the same method of measurement or calculation should be used for each of the sizes to be compared, but it is not critical which is used. Whilst Saccharomyces cerevisiae cells are generally spherical, other biological cells and the stilbenoid crystals may have an aspect ratio significantly higher than 1. In this case, it will generally be the largest dimension that is of interest in defining the particle size.

In order that a high enough proportion of the stilbenoid content of the cultivation may be present in solid form, it is preferred that the concentration of at least one said stilbenoid in the culture medium prior to said separation is at least double the solubility limit of said stilbenoid in said culture medium. Since the solubility of the stilbenoids is likely to vary with temperature, we refer here particularly to the solubility at the temperature at which separation is conducted. Optionally, said cultivation is conducted at a first temperature and said culture medium is cooled from said first temperature to a lower second temperature prior to said separation of solid stilbenoid therefrom. This may increase the amount of stilbenoid present in solid form. Suitably, the medium is cooled to no more than 10° C., e.g. no more than 5° C.

Additionally or alternatively, the culture medium may be rested to produce an increase in the average particle size of stilbenoid solids therein. A period of hours, e.g. from 6- to 24 hrs, may be allowed from the end of the cooling process or from the end of the cultivation where there is no cooling, especially from the end of any agitation.

Other steps may be taken to reduce the solubility of the stilbenoid in the medium. These may include reduction of the pH by adding an acidifying material and/or adding a salt that competes with the stilbenoid. Thus, the pH of the culture medium may be lowered to reduce the solubility of the stilbenoid such that the pH may be less than 7, e.g. below 5.5, below 4 or below 2. Once the stilbenoid has come out of solution, it would be possible to raise the pH again before actually separating it from the liquid. One preferred process would be that following fermentation, the pH is lowered and the medium is cooled, a period of time is allowed to pass as described, and thereafter solid stilbenoid is separated. Optionally, one may dilute with cold water or water miscible non-solvent to facilitate filtering without reducing crystal size.

The average particle size of stilbenoid solids in said medium is preferably at least 10 μm, for instance up to 100 μm, although the larger the better. The size differential between the stilbenoid crystals and the micro-organism cells can be increased to assist separation, for instance by the above steps of cooling the medium and/or allowing time for crystal growth or reducing the size of the cells such as by lysing them.

Various methods useful for separating crystals from biomass include plug flow filtration, diafiltration and the use of a disc stack centrifuge or a basket centrifuge.

As described below, we have found that pinosylvin tends to form crystals which are significantly larger than the crystals formed by resveratrol. Accordingly, the invention includes a method as described wherein the cultivation is performed to produce a first said stilbenoid in solid form having a first average particle size and a second said stilbenoid in solid form having a second average particle size different from said first average particle size, and a separation or partial separation of said first and second stilbenoids is carried out by separating solids of different particle sizes from one another. The separation may be carried out using filter media of intermediate pore size to allow one stilbenoid to pass whilst retaining the other.

This at least allows the content of one stilbenoid to be enriched, thus altering the ratio of stilbenoid content in the mixture.

Said stilbenoid may be one or more of resveratrol (only R³═OH), pinosylvin (all R groups are hydrogen) or piceatannol (only R³ and either R² or R⁴ is OH). Preferably, not more than 3 of the R groups are hydroxy. Preferably, the stilbenoid is trans.

Stilbenoid not recovered in solid form by mechanical separation may be extracted using a solvent, e.g. ethanol.

Preferably, said solvent comprises or consists of an ester, for instance ethyl acetate. Said ester is suitably of the general formula R⁶—COO—R⁷, and R⁶ is H or an aliphatic straight or branched chain hydrocarbon moiety of from 1-6 carbon atoms and R⁷ is an aliphatic straight or branched chain hydrocarbon moiety of from 2-16 carbon atoms, or a heteroatom containing hydrocarbon moiety of from 2 to 16 carbon atoms or an aromatic or heteroaromatic moiety of from 5 to 16 carbon atoms. R⁷ may have from 3 to 9 carbon atoms. R⁶ may have from 1 to 4 carbon atoms.

Said ester is preferably an octyl acetate, e.g. n-octyl acetate. Alternatives include hexyl, heptyl, nonyl and decyl acetates, formates and propionates.

Optionally, said solvent comprises or further comprises an alkane. It may consist of a said alkane and a said ester.

Said alkane may be a C₆ to C₁₆ straight or branched chain alkane, e.g. a C₉₋₁₄ alkane, e.g. a C₁₋₂ alkane. Preferably, said alkane is n-dodecane.

One option includes solvent extraction with n-hexane, followed by sequential extraction with 100% ether, acetone, methanol and water, and chromatographic purification on a silicagel column using a n-hexane/ethyl acetate (2/1) system.

The micro-organisms used may be naturally occurring, or recombinant micro-organisms.

Micro-organisms that may be employed include fungi, including both filamentous fungi and unicellular fungi such as yeasts, and bacteria. Yeasts are preferred, especially strains of S. cerevisiae.

The micro-organism may be one having an operative metabolic pathway comprising at least one enzyme activity, said pathway producing a said stilbenoid or an oligomeric or glycosidically-bound derivative thereof from a precursor aromatic acid of the general formula 2:

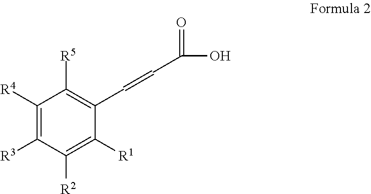

wherein each R group is as defined above.

For instance, the micro-organism may be one producing resveratrol from coumaric acid, producing pinosylvin from cinnamic acid, and/or producing piceatannol from caffeic acid.

The transformation of the said aromatic acid to the compound of Formula 1 may be by the action of an exogenous stilbene synthase expressed in said micro-organism, usually in conjunction with a suitable aromatic acid-CoA ligase serving to form the CoA thioester of the aromatic acid which together with malonyl-CoA acts as a substrate for the stilbene synthase.

Stilbene synthases are rather promiscuous enzymes that can accept a variety of physiological and non-physiological substrates. For instance, addition of various phenylpropanoid CoA starter esters led to formation of several products in vitro in Abe et al., 2004 and Morita et al., 2001. Likewise it has been shown that resveratrol synthase from rhubarb (Rheum tartaricum) indeed synthesized a small amount of pinosylvin when cinnamoyl-CoA was used as substrate instead of coumaroyl-CoA (Samappito et al., 2003).

Micro-organisms producing resveratrol for use in the invention may be as described in WO2006/089898. In particular, the micro-organism may be one having an operative metabolic pathway comprising at least one enzyme activity, said pathway producing resveratrol, or an oligomeric or glycosidically-bound derivative thereof, from 4-coumaric acid.

Micro-organisms producing pinosylvin for use in the invention may be as described in WO2008/009728 and therefore may be one that has an operative metabolic pathway comprising at least one enzyme activity, said pathway producing pinosylvin, or an oligomeric or glycosidically-bound derivative thereof, from cinnamic acid.

Malonyl-CoA for said stilbenoid forming reaction may be produced endogenously. The pool of malonyl-CoA may be increased by over expression of the gene ACC1.

The stilbene synthase may be expressed in said said micro-organism from nucleic acid coding for said enzyme which is not native to the micro-organism and may be resveratrol synthase (EC 2.3.1.95) from a plant belonging to the genus of Arachis, a plant belonging to the genus of Rheum, or a plant belonging to the genus of Vitus or any one of the genera Artocarpus, Clintonia, Morus, Vaccinium, Pinus, Picea, Lilium, Eucalyptus, Parthenocissus, Cissus, Calochortus, Polygonum, Gnetum, Artocarpus, Nothofagus, Phoenix, Festuca, Carex, Veratrum, Bauhinia or Pterolobium or may be a pinosylvin synthase (EC 2.3.1.146) from a plant belonging to the genus of Pinus, e.g. P. sylvestris, P. strobes, P. densiflora, P. taeda, a plant belonging to the genus Picea, or any one of the genus Eucalyptus.

For the preferential production of pinosylvin, the stilbene synthase may be one which exhibits a higher turnover rate with cinnamoyl-CoA as a substrate than it does with 4-coumaroyl-CoA as a substrate, e.g. by a factor of at least 1.5 or at least 2. Thus, in further preferred embodiments, said stilbene synthase is a pinosylvin synthase, suitably from a tree species such as a species of Pinus, Eucalyptus, Picea or Maclura. In particular, the stilbene synthase may be a pinosylvin synthase (EC 2.3.1.146) from a plant belonging to the genus of Pinus, e.g. P. sylvestris, P. strobes, P. densiflora, P. taeda, a plant belonging to the genus of Picea, or any one of the genus Eucalyptus.

The aromatic acid precursor may be produced in the micro-organism or may be supplied externally thereto, production by the micro-organism generally being preferred. Such aromatic acid precursors are generally producible in the micro-organism from a suitable amino acid precursor by the action of an enzyme such as a phenylalanine ammonia lyase or tyrosine ammonia lyase. The genes for the production of these enzymes may be recombinantly expressed in the micro-organism.

Thus, in certain preferred embodiments, said L-phenylalanine ammonia lyase is a L-phenylalanine ammonia lyase (EC 4.3.1.5) from a plant or a micro-organism. The plant may belong to the genus of Arabidopsis, e.g. A. thaliana, a plant belonging to the genus of Brassica, e.g. B. napes, B. rapa, a plant belonging to the genus of Citrus, e.g. C. reticulata, C. clementines, C. limon, a plant belonging to the genus of Phaseolus, e.g. P. coccineus, P. vulgaris, a plant belonging to the genus of Pinus, e.g. P. banksiana, P. monticola, P. pinaster, P. sylvestris, P. taeda, a plant belonging to the genus of Populus, e.g. P. balsamifera, P. deltoides, P. Canadensis, P. kitakamiensis, P. tremuloides, a plant belonging to the genus of Solanum, e.g. S. tuberosum, a plant belonging to the genus of Prunus, e.g. P. avium, P. persica, a plant belonging to the genus of Vitus, e.g. Vitus vinifera, a plant belonging to the genus of Zea, e.g. Z. mays or other plant genera e.g. Agastache, Ananas, Asparagus, Bromheadia, Bambusa, Beta, Betula, Cucumis, Camellia, Capsicum, Cassia, Catharanthus, Cicer, Citrullus, Coffea, Cucurbita, Cynodon, Daucus, Dendrobium, Dianthus, Digitalis, Dioscorea, Eucalyptus, Gallus, Ginkgo, Glycine, Hordeum, Helianthus, Ipomoea, Lactuca, Lithospermum, Lotus, Lycopersicon, Medicago, Malus, Manihot, Medicago, Mesembryanthemum, Nicotiana, Olea, Oryza, Pisum, Persea, Petroselinum, Phalaenopsis, Phyllostachys, Physcomitrella, Picea, Pyrus, Quercus, Raphanus, Rehmannia, Rubus, Sorghum, Sphenostylis, Stellaria, Stylosanthes, Triticum, Trifolium, Triticum, Vaccinium, Vigna, Zinnia. The micro-organism might be a fungus belonging to the genus Agaricus, e.g. A. bisporus, a fungus belonging to the genus Aspergillus, e.g. A. oryzae, A. nidulans, A. fumigatus, a fungus belonging to the genus Ustilago, e.g. U. maydis, a bacterium belonging to the genus Rhodobacter, e.g. R. capsulatus, a bacterium belonging to the genus Streptomyces, e.g. S. maritimus, a bacterium belonging to the genus Photorhabdus, e.g. P. luminescens, a yeast belonging to the genus Rhodotorula, e.g. R. rubra.

A suitable tyrosine ammonia lyase (EC 4.3.1.5) may be derived from yeast or bacteria. Suitably, the tyrosine ammonia lyase is from the yeast Rhodotorula rubra or from the bacterium Rhodobacter capsulatus.

Where the immediate product of the conversion of amino acid to aromatic acid is an aromatic acid that is not suitable as the immediate precursor of the desired stilbenoid, it may be converted to a more appropriate aromatic acid enzymatically by the micro-organism. For instance, cinammic acid may be converted to coumaric acid by a cinnamate-4-hydroxylase (C4H). Thus, said 4-coumaric acid may be produced from trans-cinnamic acid by a cinnamate 4-hydroxylase, which preferably is expressed in said micro-organism from nucleic acid coding for said enzyme which is not native to the micro-organism.

In certain preferred embodiments, said cinnamate-4-hydroxylase is a cinnamate-4-hydroxylase (EC 1.14.13.11) from a plant or a micro-organism. The plant may belong to the genus of Arabidopsis, e.g. A. thaliana, a plant belonging to the genus of Citrus, e.g. C. sinensis, C. x paradisi, a plant belonging to the genus of Phaseolus, e.g. P. vulgaris, a plant belonging to the genus of Pinus, e.g. P. taeda, a plant belonging to the genus of Populus, e.g. P. deltoides, P. tremuloides, P. trichocarpa, a plant belonging to the genus of Solanum, e.g. S. tuberosum, a plant belonging to the genus of Vitus, e.g. Vitus vinifera, a plant belonging to the genus of Zea, e.g. Z. mays, or other plant genera e.g. Ammi, Avicennia, Camellia, Camptotheca, Catharanthus, Glycine, Helianthus, Lotus, Mesembryanthemum, Physcomitrella, Ruta, Saccharum, Vigna. The micro-organism might be a fungus belonging to the genus Aspergillus, e.g. A. oryzae.

The conversion of the aromatic acid precursor into its CoA derivative may be performed by a suitable endogenous or recombinantly expressed enzyme. Both cinnamoyl-CoA and coumaroyl-CoA may be formed in a reaction catalysed by an enzyme in which ATP and CoA are substrates and ADP is a product by a 4-coumarate-CoA ligase (also referred to as 4-coumaroyl-CoA ligase). Known 4-coumarate-CoA ligase enzymes accept either 4-coumaric acid or cinnamic acid as substrates and produce the corresponding CoA derivatives. Generally, such enzymes are known as ‘4-coumarate-CoA ligase’ whether they show higher activity with 4-coumaric acid as substrate or with cinnamic acid as substrate. However, we refer here to enzymes having that substrate preference as ‘cinnamate-CoA ligase’ enzymes (or cinnamoyl-CoA-ligase). One such enzyme is described for instance in Kaneko et al., 2003.

Said 4-coumarate-CoA ligase or cinnamate-CoA ligase may be a 4-coumarate-CoA ligase/cinnamate-CoA ligase (EC 6.2.1.12) from a plant, a micro-organism or a nematode. The plant may belong to the genus of Abies, e.g. A. beshanzuensis, B. firma, B. holophylla, a plant belonging to the genus of Arabidopsis, e.g. A. thaliana, a plant belonging to the genus of Brassica, e.g. B. napes, B. rapa, B. oleracea, a plant belonging to the genus of Citrus, e.g. C. sinensis, a plant belonging to the genus of Larix, e.g. L. decidua, L. gmelinii, L. griffithiana, L. himalaica, L. kaempferi, L. laricina, L. mastersiana, L. occidentalis, L. potaninii, L. sibirica, L. speciosa, a plant belonging to the genus of Phaseolus, e.g. P. acutifolius, P. coccineus, a plant belonging to the genus of Pinus, e.g. P. armandii P. banksiana, P. pinaster, a plant belonging to the genus of Populus, e.g. P. balsamifera, P. tomentosa, P. tremuloides, a plant belonging to the genus of Solanum, e.g. S. tuberosum, a plant belonging to the genus of Vitus, e.g. Vitus vinifera, a plant belonging to the genus of Zea, e.g. Z. mays, or other plant genera e.g. Agastache, Amorpha, Cathaya, Cedrus, Crocus, Festuca, Glycine, Juglans, Keteleeria, Lithospermum, Lolium, Lotus, Lycopersicon, Malus, Medicago, Mesembryanthemum, Nicotiana, Nothotsuga, Oryza, Pelargonium, Petroselinum, Physcomitrella, Picea, Prunus, Pseudolarix, Pseudotsuga, Rosa, Rubus, Ryza, Saccharum, Suaeda, Thellungiella, Triticum, Tsuga. The micro-organism might be a filamentous fungi belonging to the genus Aspergillus, e.g. A. flavus, A. nidulans, A. oryzae, A. fumigatus, a filamentous fungus belonging to the genus Neurospora, e.g. N. crassa, a fungus belonging to the genus Yarrowia, e.g. Y. lipolytica, a fungus belonging to the genus of Mycosphaerella, e.g. M. graminicola, a bacterium belonging to the genus of Mycobacterium, e.g. M. bovis, M. leprae, M. tuberculosis, a bacterium belonging to the genus of Neisseria, e.g. N. meningitidis, a bacterium belonging to the genus of Streptomyces, e.g. S. coelicolor, a bacterium belonging to the genus of Rhodobacter, e.g. R. capsulatus, a nematode belonging to the genus Ancylostoma, e.g. A. ceylanicum, a nematode belonging to the genus Caenorhabditis, e.g. C. elegans, a nematode belonging to the genus Haemonchus, e.g. H. contortus, a nematode belonging to the genus Lumbricus, e.g. L. rubellas, a nematode belonging to the genus Meilodogyne, e.g. M. hapla, a nematode belonging to the genus Strongyloidus, e.g. S. rattii, S. stercoralis, a nematode belonging to the genus Pristionchus, e.g. P. pacificus.

Optionally, one may express, over express, or recombinantly express in said organism an NADPH:cytochrome P450 reductase (CPR). This may be a plant CPR. Alternatively, a native NADPH:cytochrome P450 reductase (CPR) may be overexpressed in said micro-organism. Optionally, said NADPH:cytochrome P450 reductase is a NADPH:cytochrome P450 reductase (EC 1.6.2.4) from a plant belonging to the genus of Arabidopsis, e.g. A. thaliana, a plant belonging to the genus of Citrus, e.g. C. sinensis, C. x paradisi, a plant belonging to the genus of Phaseolus, e.g. P. vulgaris, a plant belonging to the genus of Pinus, e.g. P. taeda, a plant belonging to the genus of Populus, e.g. P. deltoides, P. tremuloides, P. trichocarpa, a plant belonging to the genus of Solanum, e.g. S. tuberosum, a plant belonging to the genus of Vitus, e.g. Vitus vinifera, a plant belonging to the genus of Zea, e.g. Z. mays, or other plant genera e.g. Ammi, Avicennia, Camellia, Camptotheca, Catharanthus, Glycine, Helianthus, Lotus, Mesembryanthemum, Physcomitrella, Ruta, Saccharum, Vigna.

To encourage conversion of less hydroxylated stilbenoids to more hydroxylated stilbenoids, e.g. of pinosylvin to resveratrol, one may express in the micro-organism an exogenous cytochrome having the ability to accept the less hydroxylated stilbenoid as a substrate, e.g. a mammalian cytochrome P450 such as CYP2C19.

Because, as described above, for the production of pinosylvin, production of cinnamic acid by a PAL enzyme and also its conversion on to pinosylvin is preferred to either the production of coumaric acid from tyrosine by a substrate promiscuous PAL or by conversion of cinnamic acid by a C4H enzyme, micro-organisms for use in the invention to produce pinosylvin preferably have a PAL which favours phenylalanine as a substrate (thus producing cinnamic acid) over tyrosine (from which it would produce coumaric acid). Preferably, therefore, the ratio Kₘ(phenylalanine)/Kₘ(tyrosine) for the PAL is less than 1:1, preferably less 1:5, e.g. less than 1:10. As usual, Kₘ is the molar concentration of the substrate (phenylalanine or tyrosine respectively) that produces half the maximal rate of product formation (Vₘₐₓ).

In the present context the term “micro-organism” relates to microscopic organisms, including bacteria, microscopic fungi, including yeast. More specifically, the micro-organism may be a fungus, and more specifically a filamentous fungus belonging to the genus of Aspergillus, e.g. A. niger, A. awamori, A. oryzae, A. nidulans, a yeast belonging to the genus of Saccharomyces, e.g. S. cerevisiae, S. kluyveri, S. bayanus, S. exiguus, S. sevazzi, S. uvarum, a yeast belonging to the genus Kluyveromyces, e.g. K. lactis K. marxianus var. marxianus, K. thermotolerans, a yeast belonging to the genus Candida, e.g. C. utilis C. tropicalis, C. albicans, C. lipolytica, C. versatilis, a yeast belonging to the genus Pichia, e.g. P. stipidis, P. pastoris, P. sorbitophila, or other yeast genera, e.g. Cryptococcus, Debaromyces, Hansenula, Pichia, Yarrowia, Zygosaccharomyces or Schizosaccharomyces. Concerning other micro-organisms a non-exhaustive list of suitable filamentous fungi is: a species belonging to the genus Penicillium, Rhizopus, Fusarium, Fusidium, Gibberella, Mucor, Mortierella, and Trichoderma.

Concerning bacteria a non-exhaustive list of suitable bacteria is follows: a species belonging to the genus Bacillus, a species belonging to the genus Escherichia, a species belonging to the genus Lactobacillus, a species belonging to the genus Lactococcus, a species belonging to the genus Corynebacterium, a species belonging to the genus Acetobacter, a species belonging to the genus Acinetobacter, a species belonging to the genus Pseudomonas, etc.

The preferred micro-organisms of the invention may be S. cerevisiae, A. niger, A. nidulans, A. oryzae, E. coli, L. lactis or B. subtilis.

The constructed and engineered micro-organism can be cultivated using commonly known processes, including chemostat, batch, fed-batch cultivations, etc.

The micro-organism may be fed with a carbon substrate which is optionally selected from the group of fermentable carbon substrates consisting of monosaccharides, oligosaccharides and polysaccharides, e.g. glucose, fructose, galactose, xylose, arabinose, mannose, sucrose, lactose, erythrose, threose, and/or ribose. Said carbon substrate may additionally or alternatively be selected from the group of non-fermentable carbon substrates including ethanol, acetate, glycerol, and/or lactate. Said non-fermentable carbon substrate may additionally or alternatively be selected from the group of amino acids and may be phenylalanine and/or tyrosine.

The invention will be further described and illustrated by the following examples with reference to the accompanying drawings in which:

FIG. 1 shows fused divergent TEF1-TDH3 promoters used in Example 6;

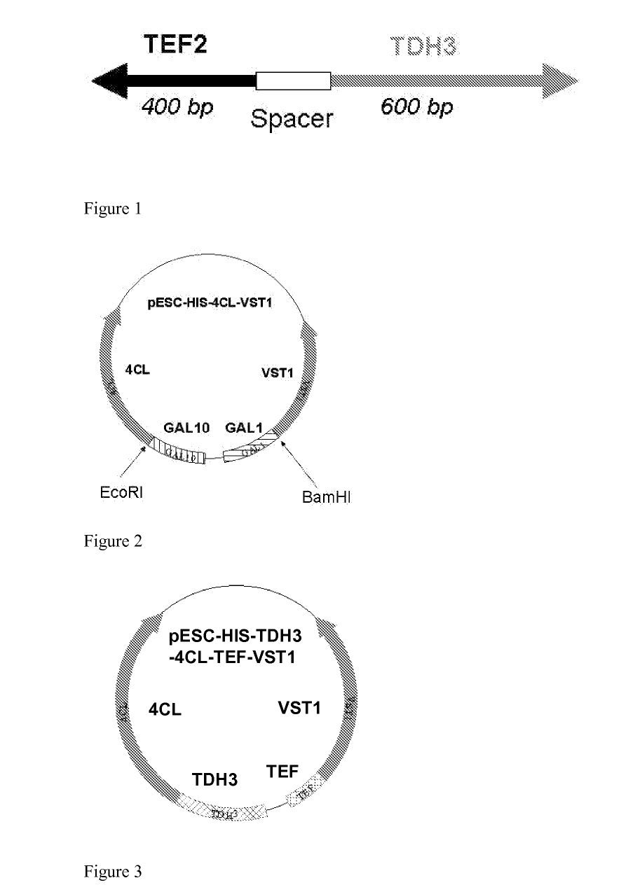

FIG. 2 shows a vector used in Example 7;

FIG. 3 shows the vector pesc-HIS-TDH3-4CL2-TEF-VST1 produced in Example 7; and

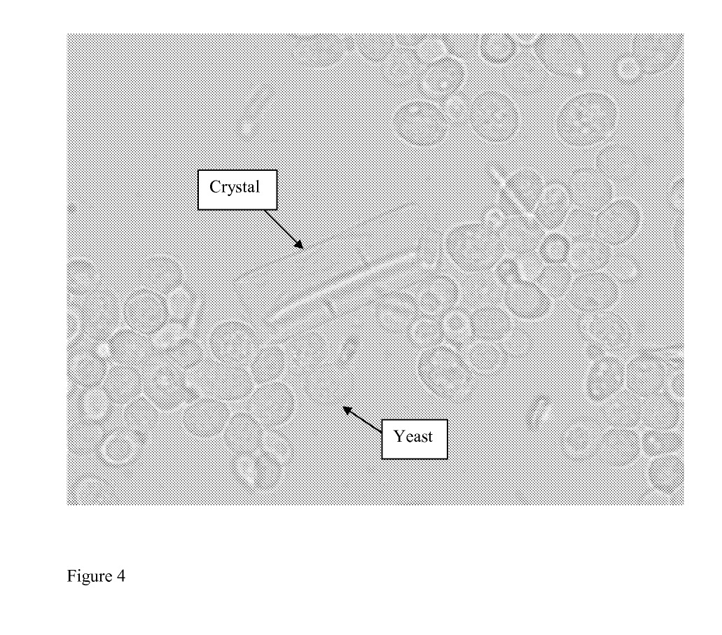

FIG. 4 is shows a stilbenoid crystal surrounded by yeast cells as seen by microscopy of the culture medium in Example 11. The pictures were captured on a cooled Evolution QEi monochrome digital camera (Media Cypernetics Inc, USA) mounted on a Nikon Eclipse E1000 microscope (Nikon, Japan).

### EXAMPLE 1

### Isolation of Genes Encoding PAL C4H, 4CL2 and VST1

4-coumarate:CoenzymeA ligase (4CL2) (Hamberger and Hahlbrock 2004; Ehlting et al., 1999; SEQ ID NO: 1) was isolated via PCR from A. thaliana cDNA (BioCat, Heidelberg, Germany) using the primers in table 1.

The PAL2 gene encoding Arabidopsis thaliana resveratrol phenylalanine ammonia lyase (Cochrane et al., 2004) was synthesized by GenScript Corporation (Piscataway, N.J.). The amino acid sequence (SEQ ID NO: 2) was used as template to generate a synthetic gene codon optimized for expression in S. cerevisiae. The synthetic PAL2 gene was delivered inserted in E. coli pUC57 vector. The synthetic gene was purified from the pUC57 vector by amplifying it by forward primer 5-CAC TAA AGG GCG GCC GCA TGG ACC AAA TTG AAG CA-3 SEQ ID NO: 11 and reverse primer 5-AAT TAA GAG CTC AGA TCT TTA GCA GAT TGG AAT AGG TG-3 SEQ ID NO: 12 and purified from agarose gel using the QiaQuick Gel Extraction Kit (Qiagen).

The C4H gene encoding Arabidopsis thaliana cinnamate-4-hydroxylase (Hamberger and Hahlbrock 2004; Ehlting et al., 1999) was synthesized by GenScript Corporation (Piscataway, N.J.). The amino acid sequence (SEQ ID NO: 3) was used as template to generate a synthetic gene codon optimized for expression in S. cerevisiae. The synthetic C4H gene was delivered inserted in E. coli pUC57 vector. The synthetic gene was purified from the pUC57 vector by amplifying it by forward primer 5-ATT TCC GAA GAA GAC CTC GAG ATG GAT TTG TTA TTG CTG G-3 SEQ ID NO:13 and reverse primer 5-AGT AGA TGG AGT AGA TGG AGT AGA TGG AGT AGA TGG ACA ATT TCT GGG TTT CAT G-3 SEQ ID NO:14 and purified from agarose gel using the QiaQuick Gel Extraction Kit (Qiagen).

The ATR2 gene encoding Arabidopsis thaliana P450 reductase was synthesized by GenScript Corporation (Piscataway, N.J.). The amino acid sequence (SEQ ID NO: 4) was used as template to generate a synthetic gene codon optimized for expression in S. cerevisiae. The synthetic C4H gene was delivered inserted in E. coli pUC57 vector. The synthetic gene was purified from the pUC57 vector by amplifying it by forward primer 5-CCA TCT ACT CCA TCT ACT CCA TCT ACT CCA TCT ACT AGG AGG AGC GGT TCG G-3 SEQ ID NO:15 and reverse primer 5-ATC TTA GCT AGC CGC GGT ACC TTA CCA TAC ATC TCT CAG ATA TC-3 SEQ ID NO:16 and purified from agarose gel using the QiaQuick Gel Extraction Kit (Qiagen).

The VST1 gene encoding Vitis vinifera (grapevine) resveratrol synthase (Hain et al., 1993) was synthesized by GenScript Corporation (Piscataway, N.J.). The amino acid sequence (SEQ ID NO: 5) was used as template to generate a synthetic gene codon optimized for expression in S. cerevisiae. The synthetic VST1 gene was delivered inserted in E. coli pUC57 vector flanked by BamH1 and Xho1 restriction sites. The synthetic gene was amplified using forward primer 5-CCG GAT CCT CAT GGC ATC CGT CGA AGA GTT CAG G-3 SEQ ID NO:17 and reverse primer 5-CGC TCG AGT TTT AGT TAG TAA CTG TGG GAA CGC TAT GC-3 SEQ ID NO:18 and purified from agarose gel using the QiaQuick Gel Extraction Kit (Qiagen).

### EXAMPLE 2

### Construction of a Yeast Vector for Galactose Induced Expression of 4CL2 and VST1

The gene encoding 4CL2 was isolated as described in example 1. The amplified 4CL2 PCR-product using forward primer 5-GCG AAT TCT TAT GAC GAC ACA AGA TGT GAT AGT CAA TGA T-3 SEQ ID NO:19 and reverse primer 5-GCA CTA GTA TCC TAG TTC ATT AAT CCA TTT GCT AGT CTT GC-3 SEQ ID NO:20 was digested with EcoR1/Spe1 and ligated into EcoR1/Spe1 digested pESC-HIS vector (Stratagene), resulting in vector pESC-HIS-4CL2.

Two different clones of pESC-HIS-4CL2 were sequenced to verify the sequence of the cloned gene.

The gene encoding VST1 was isolated as described in example 1. The amplified synthetic VST1 gene was digested with BamH1/Xho1 and ligated into BamH1/Xho1 digested pESC-HIS-4CL2. The resulting plasmid, pESC-HIS-4CL2-VST1, contained the genes encoding 4CL2 and VST1 under the control of the divergent galactose induced <=GAL1/GAL10=> promoters. The sequence of the gene encoding VST1 was verified by sequencing of two different clones of pESC-HIS-4CL2-VST1 (SEQ ID NO: 6).

### EXAMPLE 3

### Construction of a Yeast Vector for Galactose Induced Expression of PAL2 and C4H:ATR2 Fusion Gene

The gene encoding PAL2 was isolated as described in example 1. The amplified PAL2 PCR-product was inserted into NotI/BglII digested pESC-URA vector Stratagene), resulting in vector pESC-URA-PAL2. Two different clones of pESC-URA-PAL2 were sequenced to verify the sequence of the cloned gene.

The gene encoding C4H and ATR2 were isolated as described in example 1. C4H was amplified using forward primer 5-ATT TCC GAA GAA GAC CTC GAG ATG GAT TTG TTA TTG CTG G-3 SEQ ID NO:21 and reverse primer 5-AGT AGA TGG AGT AGA TGG AGT AGA TGG AGT AGA TGG ACA ATT TCT GGG TTT CAT G-3 SEQ ID NO:22. ATR2 was amplified using forward primer 5-CCA TCT ACT CCA TCT ACT CCA TCT ACT CCA TCT ACT AGG AGG AGC GGT TCG G-3 SEQ ID NO:23 and reverse primer 5-ATC TTA GCT AGC CGC GGT ACC TTA CCA TAC ATC TCT CAG ATA TC-3 SEQ ID NO:24.

The amplified PCR products C4H and ATR2 were used as templates for the creation of the fusion gene C4H:ATR2 using the forward primer 5-ATT TCC GAA GAA GAC CTC GAG ATG GAT TTG TTA TTG CTG G-3 SEQ ID NO:25 and the reverse primer 5-ATC TTA GCT AGC CGC GGT ACC TTA CCA TAC ATC TCT CAG ATA TC-3 SEQ ID NO:26.

The Fusion gene C4H:ATR2 gene was inserted into XhoI/KpnI digested pESC-URA-PAL2 by Infusion™ technology (stratagene, La jolla, USA). The resulting plasmid, pESC-URA-PAL2-C4H:ATR2, contained the genes encoding PAL2 and C4H:ATR2 under the control of the divergent galactose induced <=GAL1/GAL10=> promoters. The sequence of the gene encoding C4H:ATR2 was verified by sequencing of two different clones of pESC-URA-PAL2-C4H:ATR2 (SEQ ID NO: 7).

### EXAMPLE 4

### Construction of Strong Constitutive Promoter Fragment Tdh3

The 600 base pair TDH3 (GPD) promoter was amplified from S. cerevisiae genomic DNA using the forward primer 5′GC GAGCTC AGT TTA TCA TTA TCA ATA CTC GCC ATT TCA AAG SEQ ID NO:27 containing a Sac1 restriction site and the reverse primer 5′-CG TCTAGA ATC CGT CGA AAC TAA GTT CTG GTG TTT TAA AAC TAA AA SEQ ID NO:28 containing a Xba1 restriction site. The amplified TDH3 fragment was digested with Sac1/Xba1 and ligated into Sac1/Xba1 digested plasmid pRS416 (Sikorski and Hieter, 1989) as described previously (Mumberg et al, 1995) resulting in plasmid pRS416-TDH3.

### EXAMPLE 5

### Construction of Constitutive Strong Promoter Fragment TEF1

The 400 base pair TEF1 promoter was amplified from S. cerevisiae genomic DNA using the forward primer 5′-GC GAGCTC ATA GCT TCA AAA TGT TTC TAC TCC TTT TTT ACT CTT SEQ ID NO:29 containing a Sac1 restriction site and the reverse primer 5′-CG TCTAGA AAA CTT AGA TTA GAT TGC TAT GCT TTC TTT CTA ATG A SEQ ID NO:30 containing a Xba1 restriction site. The amplified TEF1 fragment was digested with Sac1/Xba1 and ligated into Sac1/Xba1 digested plasmid pRS416 (Sikorski and Hieter, 1989) as described previously (Mumberg et al, 1995) resulting in plasmid pRS416-TEF1.

### EXAMPLE 6

### Construction of Fused Divergent Constitutive TEF1 and TDH3 Promoter Fragment

A divergent fusion fragment (FIG. 1) between TEF1 promoter and TDH3 promoter was constructed starting from PRS416-TEF1 and PRS416-TDH3.

The 600 base pair TDH3 fragment was reamplified from PRS416-TDH3 using the forward primer 5′ TTGCGTATTGGGCGCTCTTCC GAG CTC AGT TTA TCA TTA TCA ATA CTC GC SEQ ID NO:31 containing the underlined overhang for fusion PCR to TEF1 fragment and the reverse primer 5′ AT GGATCC TCT AGA ATC CGT CGA AAC TAA GTT CTG SEQ ID NO:32 containing the underlined BamH1 restriction site. This resulted in a fragment ready for fusion to the below TEF1 fragment.

The 400 base pair TEF1 fragment including a 277 base pair spacer upstream of the Sac1 restriction site was reamplified from PRS416-TEF1 using the forward primer 5′ AT GAATTC TCT AGA AAA CTT AGA TTA GAT TGC TAT GCT TTC SEQ ID NO:33 containing the underlined EcoR1 restriction site and the reverse primer 5′ TGA TAA TGA TAA ACT GAG CTC GGA AGA GCG CCC AAT ACG CAA AC SEQ ID NO:34 containing the underlined overhang for fusion to the TDH3 fragment. This resulted in a 680 base pair fragment ready for fusion to the TDH3 fragment.

The 600 base pair TEF1 fragment and the 600 base pair TDH3 fragments were joined together (fused) using fusion PCR with the forward primer 5′ AT GAATTC TCT AGA AAA CTT AGA TTA GAT TGC TAT GCT TTC SEQ ID NO:35 and the reverse primer 5′ AT GGATCC TCT AGA ATC CGT CGA AAC TAA GTT CTG SEQ ID NO:36, resulting in the divergent fragment <=TEF1/TDH3=> (SEQ ID NO: 8).

### EXAMPLE 7

### Construction of a Yeast Vector for Constitutive Expression Induced of 4CL2 and VST1 pesc-HIS-TDH3-4CL2-TEF-VST1

The vector pESC-HIS-4CL2-VST1 (FIG. 2) with divergent galactose inducible promoters GAL1/GAL10 was sequentially digested with EcoR1 and BamH1 to remove the GAL1/GAL10 promoters.

The divergent constitutive <=TEF1/TDH3=> promoter fragment (Sequence ID 8) was reamplified with forward primers 5′ ATGAATTC TCT AGA ATC CGT CGA AAC TAA GTT CTG SEQ ID NO:37 and reverse primers AT GGA TCC TCT AGA AAA CTT AGA TTA GAT TGC TAT GCT TTC TTT CTA A SEQ ID NO:38 to reverse the orientation of TEF and TDH3 promoters in the final construct, that is to revert construct pESC-HIS-TEF1-4CL2-TDH3-VST1 into pESC-HIS-TDH3-4CL2-TEF1-VST1. The reamplified fragment was sequentially digested with EcoR1 and BamH1 and ligated into the above vector without the GAL1/Gal10 fragment. This resulted in a vector pesc-HIS-TDH3-4CL2-TEF1-VST1 (FIG. 3) with replaced promoters, from GAL1/Gal10 to TDH3/TEF1 (SEQ ID NO: 9).

### EXAMPLE 8

### Construction of a Yeast Vector for Constitutive Expression of PAL2 and C4H:ATR2 Fusion Gene

The vector pESC-URA-PAL2-C4H:ATR2 with divergent galactose inducible promoters GAL1/GAL10 was sequentially digested with NotI and XhoI to remove the GAL1/GAL10 promoters.

The divergent constitutive <=TEF1/TDH3=> promoter fragment was re-amplified with forward primer 5-TTC CAG CAA TAA CAA ATC CAT TTT GTA TCT AGA AAA CTT AGA TTA GAT TG-3 SEQ ID NO:39 and reverse primer 5-CAT TGC TTC AAT TTG GTC CAT TTT GTA TCT AGA ATC CGT CGA AAC TAA GT-3 SEQ ID NO:40. The PCR product was sequentially inserted into the above vector without the GAL1/Gal10 fragment using Infusion™ technology (stratagene, La Jolla, USA). This resulted in a vector pESC-URA-TDH3-PAL2-TEF1-C4H:ATR2 with replaced promoters, from GAL1/Gal10 to TEF1/TDH3 (SEQ ID NO: 10).

### EXAMPLE 9

### Expression of the Pathway to Pinosylvin in the Yeast S. cerevisiae Using PAL2, C4H:ATR2, 4CL2 and VST1

Yeast strains FS01529 containing the appropriate genetic markers were transformed with the vectors described in examples 7 and 8 giving FS09226. The transformation of the yeast cell was conducted in accordance with methods known in the art by using competent cells, an alternative being for instance, electroporation (see, e.g., Sambrook et al., 1989). Transformants were selected on medium lacking uracil and histidine and streak purified on the same medium.

### EXAMPLE 10

### Fed-Batch Cultivation of a Yeast Strain Containing a Functional Phenylpropanoid Pathway that is Constitutively Expressed in the Presence of Glucose

### Strain

The strain analyzed in fed-batch cultivation was the recombinant strain FS09226.

### Growth Media

The compositions of the media for the initial batch and feed are shown in Tables 1 and 2, respectively. The composition of the stock solutions of vitamins and trace metals are presented in Tables 3 and 4.

TABLE 1
Composition of the minimal medium for the
initial batch in fed-batch cultivations.

| | Concentration |
|---|---|
| Total C source [g/l] | 100.0 |
| Glucose•H₂O [g/l] | 110.0 |
| CH₄N₂O (urea) [g/l] | 11.4 |
| KH₂PO₄ [g/l] | 15.0 |
| MgSO₄ × 7H₂O [g/l] | 2.5 |
| Vitamin solution [ml/l] (see | 5.0 |
| Table 3) | |
| Trace metal solution [ml/l] | 5.0 |
| (see Table 4) | |
| Antifoam (Sigma A-8436) [μl/l] | 50.0 |

TABLE 2
Composition of the minimal medium for
the feed in fed-batch cultivations.

| | Concentration |
|---|---|
| Total C source [g/l] | 500.0 |
| Glucose•H₂O [g/l] | 550.0 |
| KH₂PO₄ [g/l] | 9.0 |
| MgSO₄ × 7H₂O [g/l] | 5.1 |
| K₂SO₄ [g/l] | 3.5 |
| Na₂SO₄ [g/l] | 0.3 |
| Vitamin solution [ml/l] (see | 12.0 |
| Table 3) | |
| Trace metal solution [ml/l] | 10.0 |
| (see Table 4) | |
| Antifoam (Sigma A-8436) [μl/l] | 50.0 |

TABLE 3
Composition of the standard vitamin solution
used in the fed-batch cultivations.

| | Concentration |
|---|---|
| | [mg/l] |
| biotin | 0.05 |
| calcium | 1.0 |
| panthotenate | |
| nicotinic acid | 1.0 |
| myo-inositol | 25.0 |
| thiamine HCl | 1.0 |
| pyridoxal HCl | 1.0 |
| para-aminobenzoic | 0.2 |
| acid | |

TABLE 4
Composition of the standard trace metal solution
used in the fed-batch cultivations.

| | Concentration |
|---|---|
| | [mg/l] |
| EDTA | 15.0 |
| ZnSO₄ × 7H₂O | 4.5 |
| MnCl₂ × 2H₂O | 1.0 |
| CoCl₂ × 6H₂O | 0.3 |
| CuSO₄ × 5H₂O | 0.3 |
| Na₂MoO₄ × 2H₂O | 0.4 |
| CaCl₂ × 2H₂O | 4.5 |
| FeSO₄ × 7H₂O | 3.0 |
| H₃BO₃ | 1.0 |
| KI | 0.1 |

The nitrogen source for the initial batch phase of the fed-batch cultivation was urea (see Table 1), whereas in the feeding phase, ammonium hydroxide (NH4OH, 25%) was used both as the nitrogen source and the base. The base used in the initial batch phase was KOH (2 N). For both the initial batch and feeding phases, HCl (2 N) was used as the acid.

### Operating Conditions

The operating conditions used in the initial batch phase and feeding phase of the fed-batch cultivations are shown in Tables 5 and 6.

TABLE 5
Operating conditions for the initial batch
phase in fed-batch cultivations.

| Parameter | Set-point |
|---|---|
| Volume of liquid [l] | 0.5 |
| Temperature [° C.] | 30.0 |
| pH | 5.5 |
| Stirrer speed [rpm] | 1200 |
| Gas flow rate [vvm]¹ | 1.5 |
| Gas flow rate [l/min] (for 1 | 0.75 |
| l of liquid) | |

¹vvm = l gas/(l liquid × min).

### Pre-Inoculum

The bioreactors were inoculated with a glycerol stock culture to a final optical density at 600 nm (OD600) of 0.12.

TABLE 6
Operating conditions for the feeding
phase in fed-batch cultivations.

| Parameter | Set-point |
|---|---|
| Volume of liquid [l] | 0.5-1.5 |
| Temperature [° C] | 30.0 |
| pH | 5.5 |
| Stirrer speed [rpm] | 1200-1800 |
| Gas flow rate [vvm]¹ | >1.5 |
| Gas flow rate [l/min] (for 1 | 2.25 |
| l of liquid) | |

¹vvm = l gas/(l liquid × min).

### Fed-Batch Cultivation

The fed-batch cultivation was performed in a bioreactor Biostat B plus (Sartorius BBI systems), with a working volume of 2 L. The initial volume of liquid used in the cultivation was 500 ml. The total volume of feed prepared was 1 l, such that the volume of liquid in the fermentor vessel did not exceed 1.5 l.

The bioreactor was equipped with two Rushton four-blade disc turbines and baffles. Air was used for sparging the bioreactors. The concentrations of oxygen, carbon dioxide, and ethanol in the exhaust gas were monitored by a gas analyzer Innova 1313 with multiplexing. Temperature, pH, agitation, and aeration rate were controlled throughout the cultivations. The temperature was maintained at 30° C. The pH was kept at 5.5 by automatic addition of KOH (2N), in the course of the initial batch, and NH4OH (25%) and HCl (2 N), during the feeding phase. The aeration rate was set to 2.25 l/min and the stirrer speed was initially set to 1200 rpm. When the dissolved oxygen dropped to values below 60%, the stirrer speed was gradually increased to values up to 1800 rpm by means of a cascade control engaged by the dissolved oxygen. The formation of foam was controlled using a foam sensor and through the addition of an antifoam agent (Sigma A-8436).

### Feeding Profiles

Initially, the feeding rate was set to an exponential profile, such that the specific growth rate was maintained constant (μ0), according to the following equation:

$$F ( t ) = \frac{{Y}_{XS} {μ}_{0}}{{S}_{feed} - {S}_{0}} {X}_{0} {V}_{0} {e}^{{μ}_{0} t}$$

where the parameters are defined in Table 7.

When the oxygen transfer appeared to be limiting due to high concentrations of biomass, and respiro-fermentative metabolism with concomitant formation of ethanol did set in, the feeding rate profile was changed to a constant value to lower the specific growth rate; the feeding rate was decreased in steps of approximately 10% in the course of the cultivation, such that oxygen limiting conditions and ethanol formation was avoided. Said procedure aimed to maximize the biomass yield.

TABLE 7
Parameters used for calculating the feeding
profile in the fed-batch cultivation.

| Symbol | Description | Unit | Value |
|---|---|---|---|
| S_{feed} | Substrate concentration in | g/l | 500 |
| | the feed | | |
| V₀ | Volume of liquid at the | l | 0.5 |
| | start of the fed-batch | | |
| | process | | |
| X₀ | Biomass concentration at | g DW/l | 10 |
| | the start of the fed-batch | | |
| | process | | |
| S₀ | Substrate concentration at | g/l | 0 |
| | the start of the fed-batch | | |
| | process | | |
| S_{feed} | Substrate concentration in | g/l | 500 |
| | the feed | | |
| Yₛₓ | Biomass yield on substrate | g DW/g | 0.47 |
| Yₓₛ | Inverse of biomass yield on | g/g DW | 2.13 |
| | substrate | | |
| μ₀ | Specific growth rate | l/h | 0.15 |

### Cell Mass Determination

Cellular growth was monitored by measuring the optical density at 600 nm (OD600) in a spectrophotometer.

Moreover, occasionally the concentration of biomass in terms of cell dry-weight (DW) was measured. Cell dry weight was determined using nitrocellulose filters (pore size 0.45 μm, Pall Corporation). The filters were pre-dried in a microwave oven at 150 W for 10 min and subsequently weighted. A known volume of cell culture was filtered and the filter cake was washed with distilled water and dried on the filter for 15 min in a microwave oven at 150 W. The filter was weighted again to determine the cell mass concentration.

### Analysis of Extracellular Metabolites

The concentrations of sugars and by-products in the filtrates were determined by using high-pressure liquid-chromatography on a column Phenomenex (Rezex 8μ 8% H. ROA-Organic Acid, 300×7.8 mm). The system was furthermore equipped with a guard column.

### Analysis of Stilbenoids

For quantitative analysis of coumaric acid, cinnamic acid, trans-resveratrol and trans-pinosylvin, samples were subjected to separation by high-performance liquid chromatography (HPLC), using a HPLC-system from Dionex, prior to uv-diode-array detection at l=306 nm. A Phenomenex (Torrance, Calif., USA) Luna 2.5 micrometer C18 (100×2.00 mm) column was used at 60° C.

The method used a non linear S-shaped gradient of acetonitrile and millipore water (both containing 50 ppm trifluoroacetic acid), at a flow of 0.8 ml/min. The S-shaped gradient profile was from 10% to 100% acetonitrile over 5 minutes. The elution time was approximately 2.0 minutes for coumaric acid, 3.0 minutes for trans-resveratrol, 3.5 minutes for cinnamic acid and 4.4 minutes for trans-pinosylvin.

Preparation of HPLC samples was rendered by addition of ethanol (99.9%) to the cell broth to a final concentration of 50% ethanol (v/v), whirly-mixing for 30 s, centrifuging for 5 minutes at 12470×g and analysis of supernatant by HPLC. When required, the samples were diluted appropriately in water prior to HPLC analysis, such that the concentrations of stilbenoids fell within the corresponding linear ranges

### EXAMPLE 11

### Formation of Crystals in a Fermentation Broth of a Fed-Batch Cultivation of a Yeast Strain Containing a Functional Phenylpropanoid Pathway that is Constitutively Expressed in the Presence of Glucose

The cell broth at the end of the fed-batch fermentation so obtained as described in example 10, contained 553.5 mg/l resveratrol and 170.9 mg/l pinosylvin (Table 8). Said concentrations were higher than the aqueous solubility (30 mg/l for resveratrol, and lower for pinosylvin); hence crystallization was likely to occur. Indeed microscopic observation of the cell-broth, using a magnification of 400- to 1000-fold, confirmed the presence of crystals. The shape of the crystals appeared rectangular with at least two sides of unequal length. Said observation is in line with earlier reported crystallization studies that classify resveratrol in the monoclinic crystal space-group P21/c (no. 14), which typically are rectangles with unequal length in three dimensions (Caruso et al. 2004). The chemical structure of pinosylvin is very similar to resveratrol and, therefore, it is plausible that pinosylvin crystallizes in the same monoclinic space group as resveratrol. Possibly, therefore, the observed crystals could be twining-crystals that are composed of both resveratrol and pinosylvin. The size of the observed crystals varied from approximately 2-fold to 10-fold the size of the adjacent yeast cells. Assuming an average size of a Saccharomyces cerevisiae cell between 3- to 7 μm, said observations implied that the size of the crystals varied from 6- to 70 μm. For comparison, a crystallization experiment was performed with 99% pure solutions of either resveratrol or pinosylvin, dissolved to concentrations of up to 1 g/l in 50% ethanol. Aliquots of 1 ml from each solution were transferred to individual open eppendorf tubes and the ethanol subsequently evaporated at room temperature within 2 days, during which resveratrol and pinosylvin precipitated respectively. The remaining saturated aqueous emulsion was carefully decanted, and an aliquot of 5 μl was microscopically investigated, at a magnification of 400- to 1000-fold, for the presence of crystals. The resveratrol-emulsion contained indeed crystals that were similar in shape to the crystals observed in the formerly mentioned cell-broth, albeit generally smaller and more uniform in size. Apparently size, more than shape, of the crystals was susceptible to the fermentation conditions that prevailed during the crystallization process in the fermentor. The pinosylvin-emulsion contained also crystals that were rectangular in shape, though seemed to be more elongated than the resveratrol crystals. Said observation, however, supports the assumption that pinosylvin crystallizes in the same crystal space group as resveratrol.

### EXAMPLE 12

### Filtration of Crystals Out of a Fermentation Broth of a Fed-Batch Cultivation of a Yeast Strain Containing a Functional Phenylpropanoid Pathway that is Constitutively Expressed in the Presence of Glucose

To enhance formation of more and larger crystals, an aliquot of 50 ml of the fermentation broth was cooled at 4° C. for 12 hrs., and subsequently diluted 5× in 4° C. Millipore water. Next, 5 ml aliquots of the diluted cell broth were vacuum-filtered using filter papers with diameter 55 mm and pore sizes of respectively 20-25 μm (Whatman hardened filter paper 54), pore size 11 μm (Whatman filter paper 1), pore size 8 μm (Whatman filter paper 2) and pore size 6 μm (Whatman filter paper 6). The range of the pore size was chosen such that cells would be separated from stilbenoid crystals (see example 10 for cell- and crystal sizes 10), with the crystals retained on the filter. The filter was then submerged into 10 ml of 50% ethanol solution and whirly-mixed for 30 seconds in order to recover and dissolve the stilbenoids retained on the filter. In addition, the filtrate (approximately 5 ml) was collected and diluted with 5 ml of 100% ethanol to obtain a 50% ethanol solution and whirly-mixed for 30 seconds in order to recover and dissolve the stilbenoids present in the filtrate. To remove cell-debris, the 50% ethanol solution obtained from both the filtrate and the filter were centrifuged for 5 min at 12470×g. The supernatant was collected and diluted 4-fold with Millipore water and was then subjected to HPLC analysis. Despite that the pore size of the filters were chosen to be equal or larger than the average yeast cell size, a cell cake was formed on all filters, though more prominent at smaller pore sizes. This was likely due to the high cell mass concentration of the cell broth that caused overloading of the filters, leading to non-specific trapping of cells.

The effectiveness of the filters was evaluated by considering the amount of resveratrol and pinosylvin that was retained on the filter, expressed as fraction of the respective stilbenoids in the cell-broth. Indeed table 8 shows that with decreasing pore size more resveratrol and

TABLE 8
Stilbenoid content of fermentation broth, filter and filtrate

| resveratrol | | pinosylvin | | resveratrol |
|---|---|---|---|---|
| mg/l | (%) | mg/l | (%) | p̅i̅n̅o̅s̅y̅l̅v̅i̅n̅ |

Filter: 20-25 um

| | | | | | |
|---|---|---|---|---|---|
| broth | 553.4 | (100%) | 170.9 | (100%) | 3.2 |
| filter | 247.6 | (44.7%) | 108.5 | (63.5%) | 2.3 |
| filtrate | 188.3 | (34%) | 55.1 | (32.2%) | 3.4 |
| total | 435.9 | (78.8%) | 163.6 | (95.7%) | |

| | | |
|---|---|---|
| filter/filtrate | 1.3 | 2.0 |

Filter: 11 um

| | | | | | |
|---|---|---|---|---|---|
| broth | 553.4 | (100%) | 170.9 | (100%) | 3.2 |
| filter | 346 | (62.5%) | 144.4 | (84.5%) | 2.4 |
| filtrate | 118.3 | (21.4%) | 30.3 | (17.7%) | 3.9 |
| total | 464.2 | (83.9%) | 174.6 | (102.2%) | |

| | | |
|---|---|---|
| filter/filtrate | 2.9 | 4.8 |

Filter: 8 um

| | | | | | |
|---|---|---|---|---|---|
| broth | 553.4 | (100%) | 170.9 | (100%) | 3.2 |
| filter | 366.8 | (66.3%) | 145.7 | (85.2%) | 2.5 |
| filtrate | 108.5 | (19.6%) | 26.3 | (15.4%) | 4.1 |
| total | 475.3 | (85.9%) | 172 | (100.6%) | |

| | | |
|---|---|---|
| filter/filtrate | 3.4 | 5.5 |

Filter: 6 um

| | | | | | |
|---|---|---|---|---|---|
| broth | 553.4 | (100%) | 170.9 | (100%) | 3.2 |
| filter | 348.1 | (62.9%) | 139.9 | (81.9%) | 2.5 |
| filtrate | 78.1 | (14.1%) | 25.6 | (15%) | 3.1 |
| total | 426.3 | (77%) | 165.4 | (96.8%) | |

| | | |
|---|---|---|
| filter/filtrate | 4.5 | 5.5 |

pinosylvin was retained on the filters (compare %-values). The filter with pore size 20-25 um could retain 45% resveratrol and 64% pinosylvin, and the maximum amount of stilbenoids retained on the filter was already reached at a pore size of 8 um, with 66%- and 85% of resveratrol and pinosylvin retained respectively. Hence, the data implied that the size of pinosylvin crystals were bigger than resveratrol crystals, because more pinosylvin was retained. Said findings were corroborated by considering the ratio of each stilbenoid between filter and filtrate. Indeed, the ratio of resveratrol-content between the filter and the filtrate was close to 1 at a pore-size of 20-25 um, whereas the filter/filtrate ratio of pinosylvin-content was already 2 at the same pore size, indicating that at least part of the pinosylvin was retained more specifically on the filter than resveratrol. The filter/filtrate ratio for resveratrol- and pinosylvin increased further with smaller pore sizes, but were always higher for pinosylvin. The filter/filtrate ratio reached its maximum for resveratrol at pore size 6 um and for pinosylvin at pore size 8 um. The selectivity of the filters for either resveratrol or pinosylvin was further demonstrated by the change in ratios between resveratrol- and pinosylvin-content on the filter and in the filtrate, compared to that in the cell-broth. The ratio of 3.4 between resveratrol- and pinosylvin content in the filtrate at a pore size of 20-25 um was only slightly higher than the ratio of 3.2 in the cell-broth, indicating that a small fraction of pinosylvin was slightly more selectively retained on that filter than resveratrol. With decreasing pore size the ratio between resveratrol- and pinosylvin content in the filtrate increased compared to the ratio in the cell-broth, indicating that the fraction of pinosylvin that was selectively retained on the filter increased compared to the fraction of resveratrol. Indeed vice-versa the ratio between resveratrol- and pinosylvin content on the filter was smaller than in the cell broth at all pore-sizes, which confirms that more pinosylvin was selectively retained on the filter than resveratrol. However, said ratio was apparently less dependent on pore size, and could reflect a higher contribution of non-selective entrapment of both resveratrol and pinosylvin within cell-sediments, that were accumulated more prominently on filters with smaller pore size. An additional indication that pinosylvin was better retained on the filters than resveratrol, was the observation that the total amount of resveratrol recovered (filter+filtrate) was not more than 77% to 86%, whereas pinosylvin could be recovered almost to completion at all pore sizes.

The lower recovery of resveratrol was caused by the fact that losses of filtrate occurred during filtration such that less than 5 ml of filtrate was collected. Yet, the filtrates were always diluted with a 5 ml aliquot of ethanol, and hence causing a dilution of more than the 2-fold dilution of the stilbenoids in the cell-broth and on the filter. Obviously this led to an underestimation of any particular stilbenoid that was present in substantial amounts in the filtrate. Because resveratrol was less retained on the filter and therefore more present in the filtrate than pinosylvin, the dilution “error” had more effect on the recovery of resveratrol then of pinosylvin.

The general observation that the ratio between resveratrol- and pinosylvin content could be changed by filtration per se, showed that stilbenoids were retained specifically, and were not solely retained by non-specific entrapment in the accumulating cell cake. This demonstrated that filtration imposes proper selective criteria for separation of resveratrol- and pinosylvin crystals. What is more, these results imply that the crystals observed in the cell broth were a mixture of pure resveratrol- and pure pinosylvin crystals, rather than twining crystals (see example 11) that are composed of both pinosylvin and resveratrol. The fact that pinosylvin retained better on the filters than resveratrol was in line with the qualitative observation that pure pinosylvin crystals appeared to be more longitudinal and longer than resveratrol crystals (example 11).

Hence, aforementioned results showed that filtration has high potential to isolate stilbenoids from a cell-broth, and separate pinosylvin from resveratrol, provided that the cell-broth contains levels of resveratrol and pinosylvin that are above the aqueous solubility, where crystallization is likely to occur. Said high stilbenoid levels were obtained with a suitable micro-organism under suitable fermentation conditions.

Separation properties, could be optimized further by manipulating the fermentation conditions such to influence crystal-growth- and size and by choosing filters with more appropriate pore size. Furthermore, the overall separation process can be improved by additional steps, such as first separating pinosylvin from the cells and resveratrol crystals, followed by extraction of the filtrate with ethanol to recover the resveratrol.

### EXAMPLE 13

### HPLC Determination of Stilbenoids and Phenylpropanoids

For quantitative analysis of coumaric acid, cinnamic acid, trans-resveratrol and trans-pinosylvin, samples were subjected to separation by high-performance liquid chromatography (HPLC), using a HPLC-system from Dionex, prior to UV-diode-array detection at l=306 nm. A Phenomenex (Torrance, Calif., USA) Gemini C6-Phenyl, 3 micron (100×3.00 mm) column was used at 35° C. The method consisted of a linear gradient of methanol and millipore water (both containing 50 ppm trifluoroacetic acid), at a flowrate of 0.5 ml/min. The gradient profile was linear from 20% methanol to 100% methanol over 20 min. The elution times were 7.5 min. for coumaric acid, 10.1 min. for trans-resveratrol, 11.8 min. for cinnamic acid and 14.0 min for pinosylvin.

### EXAMPLE 14

### Filtration of Crystals Out of a Fermentation Broth of a Fed-Batch Cultivation of a Yeast Strain Containing a Functional Phenylpropanoid Pathway that is Constitutively Expressed in the Presence of Glucose

To enhance formation of more and larger crystals, an aliquot of 50 ml of the fermentation broth with a resveratrol content of 1131 mg/l was cooled at 4° C. for 12 hrs. Furthermore, the cell-broth was subjected to a cell-invasive procedure to reduce the number of intact cells and thereby reduce accumulation of biomass and subsequent non-specific entrapment of resveratrol on filters during the filtration step. Therefore, two 1 ml aliquots of said cell broth were transferred to two fast-prep tubes, each containing 1 ml of acid-washed glass beads. Next, the tubes were positioned into a fast-extraction instrument and subjected to five extraction cycles with the instrument settings on position 4; each cycle consisted of three extraction sequences that each lasted 25 seconds. The samples were cooled between each extraction cycle by submerging the tubes into a cooler of ice for 5 minutes. The treated cell-broth was examined microscopically; the number of intact cells appeared significantly reduced and was only approximately 20- to 30% of the amount of intact cells in the non-treated cell-broth. Hereafter the treated cell broth is referred to as cell-extract. In total, 1 ml of cell-extract could be recovered from the two extraction tubes, and 0.9 ml was added and mixed into 10 ml of cooled Millipore water with a temperature of 4° C. The resveratrol content in said solution was 77.2 mg/l, hence the recovery of resveratrol after the fast extraction treatment was 100*77.2*(10/0.9)/1131=75.8%.

Next, 5-ml aliquots of the diluted cell-extract was subjected to vacuum filtration using filter papers with diameter 55 mm and pore sizes of respectively 20-25 μm (Whatman hardened filter paper 54) and pore size 11 μm (Whatman filter paper 1). The range of the pore size was chosen such that the resveratrol crystals would be retained on the filter. The filter was then submerged into 5 ml of 50% ethanol solution and whirly-mixed for 30 seconds in order to recover and dissolve the stilbenoids retained on the filter. The submersion volume of 5 ml was similar to volume of filtered cell extract; hence, differences in concentrations would directly reflect recoveries. To remove cell-debris and filter particles, the 50% ethanol solution obtained from the filter was centrifuged for 5 min. at 12470×g. The supernatant was collected and diluted 10-fold with Millipore water and was then subjected to HPLC analysis. The filtrate was turbid and clearly contained cell-debris indicating that cell particles were not retained by the filters. Approximately 5 ml filtrate was collected and diluted 2-fold with 100% ethanol to obtain a 50% ethanol solution. Said filtrate was then whirly-mixed for 30 seconds in order to recover and dissolve the stilbenoids present in the filtrate. To remove cell-debris, the 50% ethanol solution obtained from the filtrate was centrifuged for 5 min at 12470×g. The supernatant was collected and diluted 5-fold with Millipore water and was then subjected to HPLC analysis.

The effectiveness of the filters was evaluated by considering the amount of resveratrol that was retained on the filter, expressed as fraction of the resveratrol in the non-filtered cell-extract. The filter with pore size 20-25 um could retain 7.36/77.2=9.5% resveratrol only, indeed the remainder of the resveratrol was found in the filtrate. The filter with pore size 11 μm could retain more resveratrol, to the extent of 21.7/77.2=28.1% with the rest of the resveratrol in the filtrate. Decreasing pore-size indeed seemed to improve retainment of resveratrol crystals, however, recoveries could be improved with values of lower than 30%.

Though the complete cell-extract was already used in the former experiment, further experiments using filters with smaller pore-size could still be undertaken by combining the filtrates of the former experiments. Hence a new cell-extract was obtained with a resveratrol content of 66.1 mg/l resveratrol and 11.1 mg/l has been retained in the first filter step.

Next, 5-ml aliquots of said new cell-extract was subjected to vacuum filtration using filter papers with diameter 55 mm and pore size 6 μm (Whatman filter paper 6) and pore size 2.7 μm (Whatman filter paper). Handling of filters and filtrate was similar to the aforementioned procedure. The filter with pore size 6 μm could retain 14.0/66.1=21.2%, indeed the remainder of the resveratrol was found in the filtrate. This means that in the two filter steps 25.1 mg/l or 25.1/77.2=32.5% has been retained.

The filter with pore size 2.7 μm, retained even more resveratrol with a recovery of 21.9/66.1=33.1%; the residual of the resveratrol was found in the filtrate. This means that in these two filter steps 33.0 mg/l or 33.0/77.2=42.3% has been retained. Said filtrates were turbid and clearly contained cell-debris indicating that cell particles were not retained by the filters

Decreasing pore-size indeed improved retainment of resveratrol crystals, and improved recoveries while still the culture medium passed through the filters. One further experiment with smaller pore-size, was still undertaken by combining first the filtrates of the two former experiments. Hence, a cell-extract was obtained with a resveratrol content of 47.2 mg/l resveratrol. This means that in the first two filter steps 77.2-47.2=30 mg/l has been retained.

Next, a 5-ml aliquot of said new cell-extract was subjected to vacuum filtration using a filter paper with diameter 55 mm and pore size 1.6 μm (Whatman filter paper). Handling of filter and filtrate was similar to the aforementioned procedures. Indeed retainment of resveratrol using the filter with pore size 1.6 μm was much improved and recovery was now 24.5/47.2=51.9%; the remainder of the resveratrol was found in the filtrate. This means that in the three filter steps 54.5 mg/l or 54.5/77.2=70.6% has been retained. The filtrate was still turbid and contained cell-debris, but to a slightly lesser extent than in aforementioned filtration experiments, indicating that cell particles were partly retained by the filters.

Said experiments indicated that resveratrol crystals can indeed be retained on filters that have sufficient low pore sizes, to retain resveratrol and at the same time has sufficiently high pore size to allow culture medium to pass the filter. Though recoveries were not complete, retainment of crystals was better on filters with smaller pore-sizes.

The lowest pore-size tested in said set of experiments was 1.6 μm, and more then 70% of crystals could be retained. Recovery of crystals likely can be improved by using even smaller pore sizes. Yet entrapping of biomass, lysed or not, might hamper the separation efficiency of the filters, as was already observed for the filter with pore-size 1.6 μm. The efficiency of a filter-based separation process for resveratrol crystals in a fermentation broth is can be improved by adjusting either or both of pore-size and pre-treatment of the biomass.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Caruso, F., Tanski, J., Villegas-Estrada, A., Rossi, M. (2004). Structural basis for antioxidant activity of trans-resveratrol: ab initio calculations and crystal and molecular structure. J Agric Food Chem. 52, 7279-7285.
Abe, I., Watanabe, T. and Noguchi, H. (2004). Enzymatic formation of long-chain polyketide pyrones by plant type III polyketide synthases. Phytochemistry. 6, 2447-2453.
Cochrane, F. C., Davin, L. B. and Lewis N. G. (2004). The Arabidopsis phenylalanine ammonia lyase gene family: kinetic characterization of the four PAL isoforms. Phytochemistry 65, 1557-1564.
Ehlting, J., Buttner, D., Wang, Q., Douglas, C. J., Somssich, I. E. and Kombrink, E. (1999). Three 4-coumarate:coenzyme A ligases in Arabidopsis thaliana represents two evolutionary divergent classes in angiosperms. The plant journal. 19, 9-20.
Hain, R., Reif, H. J., Krause, E., Langebartels, R., Kindl, H., Vornam, B., Wiese, W., Schmelzer, E., Schreier, P. H., Stocker, R. H. and Stenzel, K. (1993). Disease resistance results from foreign phytoalexin expression in a novel plant. Nature 361, 153-156.
Hamberger, B. and Hahlbrock, K. (2004). The 4-coumarate:CoA ligase gene family in Arabidopsis thaliana comprises one rare, sinapate-activating and three commonly occurring isoenzymes. Proc. Natl. Acad. Sci. USA. 101, 2209-2214.
Kaneko, M., Ohnishi, Y. and Horinouchi, S. Cinnamate:Coenzyme A ligase from the Filamentous Bacteria Streptomyces coelicolor A3 (2), J. Bact. January 2003, vol. 185, No. 1, p. 20-27.
Mumberg D, Müller R, Funk M. Yeast vectors for the controlled expression of heterologous proteins in different genetic backgrounds. Gene. 1995 Apr. 14; 156(1):119-22.
Samappito, S., Page, J. E., Schmidt, J., De-Eknamkul, W. and Kutchan, T. M. (2003). Aromatic and pyrone polyketides synthesized by a stilbene synthase from Rheum tataricum. Phytochemistry 62, 313-323.
Sambrook, J., Fritsch, E. F. and Maniatis, T. (1989). Molecular Cloning, A Laboratory Manual, 2d edition, Cold Spring Harbor, N.Y.
Sikorski R S, Hieter P. A system of shuttle vectors and yeast host strains designed for efficient manipulation of DNA in Saccharomyces cerevisiae. Genetics. 1989 May; 122(1):19-27

1. A method for the production of a cis- or trans-stilbenoid of the general formula 1: 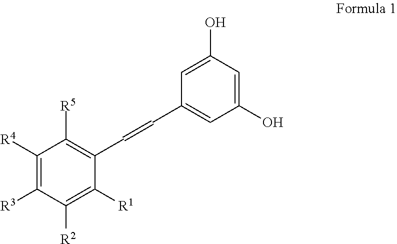
in which each of R¹, R², R³, R⁴ and R⁵ is hydrogen or hydroxy, or a glycosylated or oligomeric form thereof, comprising cultivating a micro-organism producing said stilbenoid in a culture medium, wherein said cultivation is performed so as to accumulate one or more said stilbenoids in said culture medium in solid form, and separating at least one said solid stilbenoid from the culture medium.

2. The method as claimed in claim 1, wherein said solid stilbenoid is separated from said culture medium by filtration or by settling.

3. The method as claimed in claim 1, wherein at least one said stilbenoid has an average particle size larger than cells of said micro-organism and is at least partially separated from said micro-organism cells present in said culture medium by separating solids in said culture medium according to their size.

4. The method according to claim 3, wherein the average particle size of said stilbenoid is at least double the average size of said cells.

5. The method as claimed in claim 1, wherein the concentration of at least one said stilbenoid in the culture medium prior to said separation is at least double the solubility limit of said stilbenoid in said culture medium.

6. The method as claimed in claim 1, wherein said cultivation is conducted at a first temperature and said culture medium is cooled from said first temperature to a lower second temperature prior to said separation of solid stilbenoid therefrom.

7. The method as claimed in claim 1, wherein following said cultivation, said culture medium is rested to produce an increase in the average particle size of stilbenoid solids therein.

8. The method as claimed in claim 1, wherein following said cultivation, said culture medium is acidified to produce lower the solubility of the stilbenoid therein.

9. The method as claimed in claim 8, wherein following said cultivation, the pH of said medium is lowered and/or the medium is cooled, a period of hours is allowed to pass, and thereafter solid stilbenoid is separated.

10. The method as claimed in claim 1, wherein the average particle size of stilbenoid solids in said medium is at least 10 μm.

11. The method as claimed in claim 1, wherein the cultivation is performed to produce a first said stilbenoid in solid form having a first average particle size and a second said stilbenoid in solid form having a second average particle size different from said first average particle size, and a separation or partial separation of said first and second stilbenoids is carried out by separating solids of different particle sizes from one another.


SEQUENCE LISTING (40 sequences)

SEQ ID NO: 1 - DNA, 1671 bp, Arabidopsis thaliana
atgacgacacaagatgtgatagtcaatgatcagaatgatcagaaacagtgtagtaatgacgtcattttccgatcgagattgcctgatatatacatccctaaccacctcccactccacgactacatcttcgaaaatatctcagagttcgccgctaagccatgcttgatcaacggtcccaccggcgaagtatacacctacgccgatgtccacgtaacatctcggaaactcgccgccggtcttcataacctcggcgtgaagcaacacgacgttgtaatgatcctcctcccgaactctcctgaagtagtcctcactttccttgccgcctccttcatcggcgcaatcaccacctccgcgaacccgttcttcactccggcggagatttctaaacaagccaaagcctccgcggcgaaactcatcgtcactcaatcccgttacgtcgataaaatcaagaacctccaaaacgacggcgttttgatcgtcaccaccgactccgacgccatccccgaaaactgcctccgtttctccgagttaactcagtccgaagaaccacgagtggactcaataccggagaagatttcgccagaagacgtcgtggcgcttcctttctcatccggcacgacgggtctccccaaaggagtgatgctaacacacaaaggtctagtcacgagcgtggcgcagcaagtcgacggcgagaatccgaatctttacttcaacagagacgacgtgatcctctgtgtcttgcctatgttccatatatacgctctcaactccatcatgctctgtagtctcagagttggtgccacgatcttgataatgcctaagttcgaaatcactctcttgttagagcagatacaaaggtgtaaagtcacggtggctatggtcgtgccaccgatcgttttagctatcgcgaagtcgccggagacggagaagtatgatctgagctcggttaggatggttaagtctggagcagctcctcttggtaaggagcttgaagatgctattagtgctaagtttcctaacgccaagcttggtcagggctatgggatgacagaagcaggtccggtgctagcaatgtcgttagggtttgctaaagagccgtttccagtgaagtcaggagcatgtggtacggtggtgaggaacgccgagatgaagatacttgatccagacacaggagattctttgcctaggaacaaacccggcgaaatatgcatccgtggcaaccaaatcatgaaaggctatctcaatgaccccttggccacggcatcgacgatcgataaagatggttggcttcacactggagacgtcggatttatcgatgatgacgacgagcttttcattgtggatagattgaaagaactcatcaagtacaaaggatttcaagtggctccagctgagctagagtctctcctcataggtcatccagaaatcaatgatgttgctgtcgtcgccatgaaggaagaagatgctggtgaggttcctgttgcgtttgtggtgagatcgaaagattcaaatatatccgaagatgaaatcaagcaattcgtgtcaaaacaggttgtgttttataagagaatcaacaaagtgttcttcactgactctattcctaaagctccatcagggaagatattgaggaaggatctaagagcaagactagcaaatggattaatgaactag

SEQ ID NO: 2 - DNA, 2154 bp, Arabidopsis thaliana
atggatcaaatcgaagcaatgttgtgcggcggaggagagaagacaaaagtggcggttactacgaagactttggcagatccattgaattggggtttagcagcggatcaaatgaaaggaagtcatttagatgaagtgaagaagatggtcgaagagtatcgtagaccagtcgtgaatcttggcggagaaacactgacgatcggacaagttgctgccatctccaccgtaggaggcagcgttaaggttgagttagcggagacttcaagagccggtgtgaaagctagcagtgattgggttatggagagcatgaacaaaggtactgacagttacggagtcaccaccggctttggtgctacttctcaccggagaaccaaaaacggcaccgcattacaaacagaactcattagatttttgaacgccggaatattcggaaacacgaaggagacatgtcacacactgccgcaatccgccacaagagccgccatgctcgtcagagtcaacactcttctccaaggatactccgggatccgattcgagatcctcgaagcgattacaagtctcctcaaccacaacatctctccgtcactacctctccgtggaaccattaccgcctccggcgatctcgttcctctctcttacatcgccggacttctcaccggccgtcctaattccaaagccaccggtcccgacggtgaatcgctaaccgcgaaagaagcttttgagaaagccggaatcagtactggattcttcgatttacaacctaaggaaggtttagctctcgttaatggcacggcggttggatctggaatggcgtcgatggttctattcgaagcgaatgtccaagcggtgttagcggaggttttatcagcgatcttcgcggaggttatgagcgggaaacctgagtttaccgatcatctgactcatcgtttaaaacatcatcccggacaaatcgaagcggcggcgataatggagcacatactcgacggaagctcatacatgaaattagctcaaaaggttcacgagatggatccattgcagaaaccaaaacaagatcgttacgctcttcgtacatctcctcaatggctaggtcctcaaattgaagtaatccgtcaagctacgaaatcgatagagcgtgaaatcaactccgttaacgataatccgttgatcgatgtttcgaggaacaaggcgattcacggtggtaacttccaaggaacaccaatcggagtttctatggataacacgagattggcgattgctgcgattgggaagctaatgtttgctcaattctctgagcttgttaatgatttctacaacaatggacttccttcgaatctaactgcttcgagtaatccaagtttggattatggattcaaaggagcagagattgctatggcttcttattgttctgagcttcaatacttggctaatccagtcacaagccatgttcaatcagctgagcaacataatcaagatgtgaactctcttggtttgatctcgtctcgtaaaacatctgaagctgtggatattcttaagctaatgtcaacaacgttccttgtggggatatgtcaagctgttgatttgagacatttggaggagaatctgagacaaactgtgaagaacacagtttctcaagttgctaagaaagtgttaaccactggaatcaacggtgagttacatccgtcaaggttttgcgagaaggacttgcttaaggttgttgatcgtgagcaagtgttcacgtatgtggatgatccttgtagcgctacgtacccgttgatgcagagactaagacaagttattgttgatcacgctttgtccaacggtgagactgagaagaatgcagtgacttcgatctttcaaaagattggagcttttgaagaggagcttaaggctgtgcttccaaaggaagttgaagcggctagagcggcttatgggaatggaactgcgccgattcctaaccggattaaggaatgtaggtcgtatccgttgtataggttcgtgagggaagagcttggaacgaagttgttgactggagaaaaggttgtgtctccgggagaggagtttgataaggtcttcactgctatgtgtgaaggtaaacttattgatccgttgatggattgtctcaaggaatggaacggagctccgattccgatttgctaa

SEQ ID NO: 3 - DNA, 1518 bp, artificial
  synthetic gene
atggacctcctcttgctggagaagtctttaatcgccgtcttcgtggcggtgattctcgccacggtgatttcaaagctccgcggcaagaaattgaagctacctccaggtcctataccaattccgatcttcggaaactggcttcaagtcggagatgatctcaaccaccgtaatctcgtcgattacgctaagaaattcggcgatctcttcctcctccgtatgggtcagcgaaacctagtcgtcgtctcctcaccggatctaacaaaggaagtgctcctcactcaaggcgttgagtttggatccagaacgagaaacgtcgtgttcgacattttcaccgggaaaggtcaagatatggtgttcactgtttacggcgagcattggaggaagatgagaagaatcatgacggttcctttcttcaccaacaaagttgttcaacagaatcgtgaaggttgggagtttgaagcagctagtgttgttgaagatgttaagaagaatccagattctgctacgaaaggaatcgtgttgaggaaacgtttgcaattgatgatgtataacaatatgttccgtatcatgttcgatagaagatttgagagtgaggatgatcctcttttccttaggcttaaggctttgaatggtgagagaagtcgattagctcagagctttgagtataactatggagatttcattcctatccttagaccattcctcagaggctatttgaagatttgtcaagatgtgaaagatcgaagaatcgctcttttcaagaagtactttgttgatgagaggaagcaaattgcgagttctaagcctacaggtagtgaaggattgaaatgtgccattgatcacatccttgaagctgagcagaagggagaaatcaacgaggacaatgttctttacatcgtcgagaacatcaatgtcgccgcgattgagacaacattgtggtctatcgagtggggaattgcagagctagtgaaccatcctgaaatccagagtaagctaaggaacgaactcgacacagttcttggaccgggtgtgcaagtcaccgagcctgatcttcacaaacttccataccttcaagctgtggttaaggagactcttcgtctgagaatggcgattcctctcctcgtgcctcacatgaacctccatgatgcgaagctcgctggctacgatatcccagcagaaagcaaaatccttgttaatgcttggtggctagcaaacaaccccaacagctggaagaagcctgaagagtttagaccagagaggttctttgaagaagaatcgcacgtggaagctaacggtaatgacttcaggtatgtgccatttggtgttggacgtcgaagctgtcccgggattatattggcattgcctattttggggatcaccattggtaggatggtccagaacttcgagcttcttcctcctccaggacagtctaaagtggatactagtgagaaaggtggacaattcagcttgcacatccttaaccactccataatcgttatgaaaccaaggaactgttaa

SEQ ID NO: 4 - DNA, 2136 bp, artificial
  synthetic gene
atgtcctcttcttcttcttcgtcaacctccatgatcgatctcatggcagcaatcatcaaaggagagcctgtaattgtctccgacccagctaatgcctccgcttacgagtccgtagctgctgaattatcctctatgcttatagagaatcgtcaattcgccatgattgttaccacttccattgctgttcttattggttgcatcgttatgctcgtttggaggagatccggttctgggaattcaaaacgtgtcgagcctcttaagcctttggttattaagcctcgtgaggaagagattgatgatgggcgtaagaaagttaccatctttttcggtacacaaactggtactgctgaaggttttgcaaaggctttaggagaagaagctaaagcaagatatgaaaagaccagattcaaaatcgttgatttggatgattacgcggctgatgatgatgagtatgaggagaaattgaagaaagaggatgtggctttcttcttcttagccacatatggagatggtgagcctaccgacaatgcagcgagattctacaaatggttcaccgaggggaatgacagaggagaatggcttaagaacttgaagtatggagtgtttggattaggaaacagacaatatgagcattttaataaggttgccaaagttgtagatgacattcttgtcgaacaaggtgcacagcgtcttgtacaagttggtcttggagatgatgaccagtgtattgaagatgactttaccgcttggcgagaagcattgtggcccgagcttgatacaatactgagggaagaaggggatacagctgttgccacaccatacactgcagctgtgttagaatacagagtttctattcacgactctgaagatgccaaattcaatgatataaacatggcaaatgggaatggttacactgtgtttgatgctcaacatccttacaaagcaaatgtcgctgttaaaagggagcttcatactcccgagtctgatcgttcttgtatccatttggaatttgacattgctggaagtggacttacgtatgaaactggagatcatgttggtgtactttgtgataacttaagtgaaactgtagatgaagctcttagattgctggatatgtcacctgatacttatttctcacttcacgctgaaaaagaagacggcacaccaatcagcagctcactgcctcctcccttcccaccttgcaacttgagaacagcgcttacacgatatgcatgtcttttgagttctccaaagaagtctgctttagttgcgttggctgctcatgcatctgatcctaccgaagcagaacgattaaaacaccttgcttcacctgctggaaaggatgaatattcaaagtgggtagtagagagtcaaagaagtctacttgaggtgatggccgagtttccttcagccaagccaccacttggtgtcttcttcgctggagttgctccaaggttgcagcctaggttctattcgatatcatcatcgcccaagattgctgaaactagaattcacgtcacatgtgcactggtttatgagaaaatgccaactggcaggattcataagggagtgtgttccacttggatgaagaatgctgtgccttacgagaagagtgaaaactgttcctcggcgccgatatttgttaggcaatccaacttcaagcttccttctgattctaaggtaccgatcatcatgatcggtccagggactggattagctccattcagaggattccttcaggaaagactagcgttggtagaatctggtgttgaacttgggccatcagttttgttctttggatgcagaaaccgtagaatggatttcatctacgaggaagagctccagcgatttgttgagagtggtgctctcgcagagctaagtgtcgccttctctcgtgaaggacccaccaaagaatacgtacagcacaagatgatggacaaggcttctgatatctggaatatgatctctcaaggagcttatttatatgtttgtggtgacgccaaaggcatggcaagagatgttcacagatctctccacacaatagctcaagaacaggggtcaatggattcaactaaagcagagggcttcgtgaagaatctgcaaacgagtggaagatatcttagagatgtatggtaa

SEQ ID NO: 5 - DNA, 1182 bp, artificial
  synthetic gene
atggcatccgtagaggagttcagaaatgcacagagggcaaaaggtccagcaaccatattggctattggaacagccacccctgatcactgtgtttatcaatctgattacgctgattactatttcagagtaactaaaagtgaacatatgacagaacttaagaaaaagtttaatagaatttgtgataaatctatgataaagaaaagatacatacatctaactgaagaaatgttagaggaacatccaaatataggtgcatatatggcaccatctttgaatattagacaagaaatcataacagccgaggtacctagactaggtagagacgcagccttgaaagctttaaaggaatggggacaaccaaaatctaagattacacatttggttttctgtacaacttccggtgtcgaaatgccaggtgctgattataaactagcaaacctattgggattagagacctctgttagaagagttatgttgtatcatcaaggttgttacgccggaggtacagtgcttagaactgctaaggatttggcagaaaataacgccggtgctagggttttagtcgtctgcagtgaaatcactgtcgtaactttcagaggtccatcagaagatgctctagacagtttggtcggacaagcattgtttggcgatggatcttccgccgtaattgtaggcagcgatcctgatgtgtccattgaaagaccactatttcaattagtttctgctgctcaaacttttattccaaattccgccggtgccatagcaggaaacttgagagaagttggtttgacttttcatttgtggcctaatgtcccaaccttaatttcagaaaacatcgaaaaatgcttaactcaagcctttgacccattgggcataagcgactggaactcattgttttggattgctcatccaggtggtccagcaattttagacgcagtggaggcaaaactaaacttagagaagaaaaagttggaagctacaagacacgttctatcagagtatggcaacatgagctctgcctgcgttttattcattctagatgagatgaggaagaagtctttaaagggtgaaaaagccacaaccggagaaggtttagattggggtgttctatttggtttcggtcctggcttaacaattgagacagtggtgttacactctgttccaactgtcactaactaatga

SEQ ID NO: 6 - DNA, 10157 bp, artificial
  vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtctagttcattaatccatttgctagtcttgctcttagatccttcctcaatatcttccctgatggagctttaggaatagagtcagtgaagaacactttgttgattctcttataaaacacaacctgttttgacacgaattgcttgatttcatcttcggatatatttgaatctttcgatctcaccacaaacgcaacaggaacctcaccagcatcttcttccttcatggcgacgacagcaacatcattgatttctggatgacctatgaggagagactctagctcagctggagccacttgaaatcctttgtacttgatgagttctttcaatctatccacaatgaaaagctcgtcgtcatcatcgataaatccgacgtctccagtgtgaagccaaccatctttatcgatcgtcgatgccgtggccaaggggtcattgagatagcctttcatgatttggttgccacggatgcatatttcgccgggtttgttcctaggcaaagaatctcctgtgtctggatcaagtatcttcatctcggcgttcctcaccaccgtaccacatgctcctgacttcactggaaacggctctttagcaaaccctaacgacattgctagcaccggacctgcttctgtcatcccatagccctgaccaagcttggcgttaggaaacttagcactaatagcatcttcaagctccttaccaagaggagctgctccagacttaaccatcctaaccgagctcagatcatacttctccgtctccggcgacttcgcgatagctaaaacgatcggtggcacgaccatagccaccgtgactttacacctttgtatctgctctaacaagagagtgatttcgaacttaggcattatcaagatcgtggcaccaactctgagactacagagcatgatggagttgagagcgtatatatggaacataggcaagacacagaggatcacgtcgtctctgttgaagtaaagattcggattctcgccgtcgacttgctgcgccacgctcgtgactagacctttgtgtgttagcatcactcctttggggagacccgtcgtgccggatgagaaaggaagcgccacgacgtcttctggcgaaatcttctccggtattgagtccactcgtggttcttcggactgagttaactcggagaaacggaggcagttttcggggatggcgtcggagtcggtggtgacgatcaaaacgccgtcgttttggaggttcttgattttatcgacgtaacgggattgagtgacgatgagtttcgccgcggaggctttggcttgtttagaaatctccgccggagtgaagaacgggttcgcggaggtggtgattgcgccgatgaaggaggcggcaaggaaagtgaggactacttcaggagagttcgggaggaggatcattacaacgtcgtgttgcttcacgccgaggttatgaagaccggcggcgagtttccgagatgttacgtggacatcggcgtaggtgtatacttcgccggtgggaccgttgatcaagcatggcttagcggcgaactctgagatattttcgaagatgtagtcgtggagtgggaggtggttagggatgtatatatcaggcaatctcgatcggaaaatgacgtcattactacactgtttctgatcattctgatcattgactatcacatcttgtgtcgtcatgaattctctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagaggatccatggcatccgtagaggagttcagaaatgcacagagggcaaaaggtccagcaaccatattggctattggaacagccacccctgatcactgtgtttatcaatctgattacgctgattactatttcagagtaactaaaagtgaacatatgacagaacttaagaaaaagtttaatagaatttgtgataaatctatgataaagaaaagatacatacatctaactgaagaaatgttagaggaacatccaaatataggtgcatatatggcaccatctttgaatattagacaagaaatcataacagccgaggtacctagactaggtagagacgcagccttgaaagctttaaaggaatggggacaaccaaaatctaagattacacatttggttttctgtacaacttccggtgtcgaaatgccaggtgctgattataaactagcaaacctattgggattagagacctctgttagaagagttatgttgtatcatcaaggttgttacgccggaggtacagtgcttagaactgctaaggatttggcagaaaataacgccggtgctagggttttagtcgtctgcagtgaaatcactgtcgtaactttcagaggtccatcagaagatgctctagacagtttggtcggacaagcattgtttggcgatggatcttccgccgtaattgtaggcagcgatcctgatgtgtccattgaaagaccactatttcaattagtttctgctgctcaaacttttattccaaattccgccggtgccatagcaggaaacttgagagaagttggtttgacttttcatttgtggcctaatgtcccaaccttaatttcagaaaacatcgaaaaatgcttaactcaagcctttgacccattgggcataagcgactggaactcattgttttggattgctcatccaggtggtccagcaattttagacgcagtggaggcaaaactaaacttagagaagaaaaagttggaagctacaagacacgttctatcagagtatggcaacatgagctctgcctgcgttttattcattctagatgagatgaggaagaagtctttaaagggtgaaaaagccacaaccggagaaggtttagattggggtgttctatttggtttcggtcctggcttaacaattgagacagtggtgttacactctgttccaactgtcactaactaatgactcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 7 - DNA, 12214 bp, artificial
  vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatctttagcagattggaataggtgcaccattccactctttcaagcaatccataagtggatctatcaactttccctcgcacatagctgtgaataccttgtcaaattcttcacctgggctaacgactttttcaccagttagtaatttggttcccaactcttctctaacgaatctgtacaaagggtacgacctacactctttgattctatttggtataggggcagtaccatttccgtatgcggctctagcagcttcgacttcctttggtaaaactgccttcagttcttcttcaaaggcacctatcttttggaatattgaagtaacggcatttttctcagtttcaccattggataaagcgtgatctacaataacttgtctcaatctctgcatcaatggataagtagcgctacatggatcgtcaacgtaagtaaatacttgttctctatctacaacttttaataaatctttttcacagaatcttgatgggtgcaattcaccattgatacctgtagttagaaccttttttgcaacctgtgatacggtatttttcactgtctgtctcaaattctcttccaagtgtctcaaatctacggcctggcatatacccactaaaaatgttgtggacattaatttaaggatatcaacggcctcgcttgtttttcttgatgaaatcaggcccaaagaattaacatcctgattgtgttgttcggctgattgtacatgagaggttactgggttggctagatattgcagctctgaacaatagcttgccattgctatctcagcacctttgaaaccataatcaagactagggttagaagatgcggtcagattcgaaggcaaaccgttattgtagaagtcattgaccaattcagaaaattgggcaaacattaatttgccaattgcggctatggcaagcctggtattatccatactgactcctatgggtgtaccctggaaattgcctccatgtattgccttattcctcgacacatcaataagtggattatcgttaacagagttgatctctctttctatagactttgtagcttgtctaattacttcaatttgagggccaagccattgtggggatgtccttaaagcatatctatcttgtttgggtttttgcaaagggtccatttcatgaaccttctgggctaacttcatgtagctagagccgtccaaaatgtgctccatgatagctgctgcttcaatttgtcctgggtgatgttttaacctgtgggtcaagtgatcagtaaactcaggttttccactcatgacttcggcaaaaattgcggacaaaacttcggccaaaactgcttgtacgttagcttcaaacaacaccatggatgccataccgctgccgacagcggtgccattcaccagggctaaaccttccttgggttgcaaatcaaagaaaccagttgaaataccagctttctcaaatgcttccttagcggttaaggattctccgtctggaccagtggcctttgaattaggtcttcccgttaataagcctgcgatatatgaaaggggaaccaaatcaccgctggcagttattgttcctcttaagggcaacgaaggagaaatgttgtggttcaatagtgaagtgatggcctcaagaatttcaaaccttattccagagtaaccttgcaacaaagtgttcaccctaacaagcatagcagctcttgttgccgattggggtaatgtatggcaagtttcctttgtattaccgaaaataccggcgttaaggaatctgatcagttctgtttgcaaagcagtgccatttttagttcttctatgagaggtagcaccaaagcctgtggtaacgccataggaatctgtgcccttgttcatactttccatgacccaatctgatgaagccttaactccggctctacttgtttctgcaagttctaccttcactgaaccgccaacggtcgaaatagcagctacctgtcctatcgtcaatgtctcgccgcctagatttacgactggtcttctgtattcctcaaccatcttcttaacttcatccagatggctacctttcatctggtcagctgccagaccccaattcaaaggatctgcaagagtttttgtcgttacggccaccttggtcttttcaccaccaccgcatagcattgcttcaatttggtccatgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactatagggcccgggcgtcgacatggaacagaagttgatttccgaagaagacctcgagatggatttgttattgctggaaaagtcacttattgctgtatttgtggcagttattctagccacggttatttctaaattaagaggtaagaaactaaaactacctcctggtcccatccccataccaatttttggtaattggttgcaagtgggcgatgatttgaatcacagaaatttggtagactatgctaagaagttcggtgaccttttcttgcttagaatgggtcaaaggaatttggtagtggttagctcacctgatttgactaaggaggtcttattaacgcaaggcgttgagtttggctccagaactagaaatgttgtgtttgatattttcactggtaaaggtcaagatatggtttttacagtttacggtgagcactggagaaaaatgagaagaatcatgaccgtaccattctttactaacaaggttgttcaacaaaatagagaaggttgggagtttgaggcagcttccgtagtggaagacgtaaagaaaaatccagattcggccacaaagggtatagtactaagaaaaagactacaattgatgatgtacaacaatatgttcagaattatgtttgacagaagatttgaaagtgaagatgaccctttgttcctgagacttaaggctttgaatggtgaaagatcgagattggctcaaagtttcgaatataattacggtgactttattccaatcttaagaccatttttgagaggctatttgaaaatttgccaagacgtcaaggataggaggatcgctcttttcaagaagtactttgtggacgagagaaagcaaatagcttcttccaagcccacaggttcggaaggtttaaaatgtgcaattgatcatattttagaagctgaacaaaaaggtgaaattaacgaagataatgttttgtacattgtagaaaatatcaatgtggctgcaatagaaacaaccttatggtcaatagaatggggtattgctgaattggtgaatcacccagaaatacaatctaaactgagaaacgagctagataccgttttaggtccaggtgtccaagttacagaacctgatttgcataagttaccctacttgcaagctgtggttaaagaaaccttgagattgagaatggctattcctcttctagttcctcatatgaacctacatgatgctaaactggccggttatgatattccagcagaaagtaagattttagtaaatgcatggtggttggccaacaatccaaacagttggaaaaagcctgaagaattcagacctgaaagattcttcgaagaggaatctcatgttgaagccaacggaaatgacttcagatatgtaccttttggcgttggcagaagatcgtgtccaggaataatactagccttaccaatattgggtatcacaattggtaggatggttcaaaattttgagttgctaccaccacccggacaatcgaaagtcgatacttcagagaaaggaggacaattctcattgcatattttgaatcattccattatagtcatgaaacccagaaattgtccatctactccatctactccatctactccatctactaggaggagcggttcgggcaattcaaagagggttgaaccactaaagccattagttatcaaacctagagaagaggaaattgacgatggaaggaagaaagtcactatattcttcggcacccaaacaggtacagctgaaggttttgctaaggctctaggagaagaagcaaaagctagatatgaaaagacgagattcaaaattgtcgatctggatgactatgccgccgatgatgacgaatacgaagaaaaattgaagaaagaagatgtcgcatttttcttccttgccacctacggcgacggtgaaccaacagataatgccgcaaggttttacaagtggtttactgaaggtaatgacagaggagaatggctgaagaatttgaaatatggtgtgttcggccttggtaacagacagtacgagcattttaataaggtcgctaaggttgtagatgatatacttgttgaacaaggtgctcaaaggttagtgcaggtgggcttgggtgacgatgatcaatgtattgaagatgactttactgcttggagagaagccttgtggcctgaattagatactatccttagagaagaaggtgacactgctgttgctaccccctacactgcagcagtcctagaatatagagtctcaatccatgattcagaagacgccaaattcaatgatattaacatggccaacggtaacggttacaccgtttttgacgcacaacatccatacaaagctaatgttgctgttaaaagggaacttcacaccccagaaagtgacaggtcatgtatacatttggaatttgatatcgctggtagtggtttgacttacgaaacaggtgaccatgtcggagtactttgcgataatttgtcagaaactgttgatgaagctttgaggttattggatatgtcaccagatacttacttctcattgcatgcagaaaaagaagacggaactccaatatcaagctcgcttccccctccattccctccctgtaacttaagaacagccctaactagatatgcttgtttactgtcttctccaaagaaaagtgctttggttgcattggcagcccacgcatccgatcctaccgaagctgagagattaaagcatttggcttcaccagccggtaaagatgaatacagtaagtgggtagtggagagccaaagatcgcttttagaagtgatggctgagtttccaagtgctaaacctcctctgggtgtatttttcgctggtgtggccccaagattgcagcctagattttattccatatcctcatctccaaaaattgccgaaaccagaattcacgtgacatgtgctctggtctacgaaaagatgccaacaggtaggattcacaagggtgtctgttctacctggatgaaaaatgctgtaccctatgaaaaatccgaaaattgttctagtgcaccaattttcgtaagacaatctaatttcaagttaccaagcgattctaaagtacccattattatgatcggtccaggtactggtttggccccattcagaggcttcttgcaagaaagattggctttagtggagagtggagttgaattgggtccttcagttttattctttggttgtagaaacagaagaatggactttatctacgaagaagaattgcagagatttgttgaaagtggtgcattggccgaattgagtgttgcattcagcagggaaggtccaaccaaagaatacgttcaacacaagatgatggacaaggcttctgatatctggaatatgatttcccaaggtgcttatttgtatgtttgtggtgacgctaaaggaatggctagagatgttcatagatcactgcatacaatcgcacaagaacaaggtagcatggattcaacaaaagcagagggctttgtaaagaatcttcagacaagcggtagatatctgagagatgtatggtaaggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 8 - DNA, 1373 bp, artificial
  fused promoter fragment
atgaattctctagaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaggatccat

SEQ ID NO: 9 - DNA, 10157 bp, artificial; identical to SEQ ID NO: 6
  vector

SEQ ID NO: 10 - DNA, 12806 bp, artificial
  vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatctttagcagattggaataggtgcaccattccactctttcaagcaatccataagtggatctatcaactttccctcgcacatagctgtgaataccttgtcaaattcttcacctgggctaacgactttttcaccagttagtaatttggttcccaactcttctctaacgaatctgtacaaagggtacgacctacactctttgattctatttggtataggggcagtaccatttccgtatgcggctctagcagcttcgacttcctttggtaaaactgccttcagttcttcttcaaaggcacctatcttttggaatattgaagtaacggcatttttctcagtttcaccattggataaagcgtgatctacaataacttgtctcaatctctgcatcaatggataagtagcgctacatggatcgtcaacgtaagtaaatacttgttctctatctacaacttttaataaatctttttcacagaatcttgatgggtgcaattcaccattgatacctgtagttagaaccttttttgcaacctgtgatacggtatttttcactgtctgtctcaaattctcttccaagtgtctcaaatctacggcctggcatatacccactaaaaatgttgtggacattaatttaaggatatcaacggcctcgcttgtttttcttgatgaaatcaggcccaaagaattaacatcctgattgtgttgttcggctgattgtacatgagaggttactgggttggctagatattgcagctctgaacaatagcttgccattgctatctcagcacctttgaaaccataatcaagactagggttagaagatgcggtcagattcgaaggcaaaccgttattgtagaagtcattgaccaattcagaaaattgggcaaacattaatttgccaattgcggctatggcaagcctggtattatccatactgactcctatgggtgtaccctggaaattgcctccatgtattgccttattcctcgacacatcaataagtggattatcgttaacagagttgatctctctttctatagactttgtagcttgtctaattacttcaatttgagggccaagccattgtggggatgtccttaaagcatatctatcttgtttgggtttttgcaaagggtccatttcatgaaccttctgggctaacttcatgtagctagagccgtccaaaatgtgctccatgatagctgctgcttcaatttgtcctgggtgatgttttaacctgtgggtcaagtgatcagtaaactcaggttttccactcatgacttcggcaaaaattgcggacaaaacttcggccaaaactgcttgtacgttagcttcaaacaacaccatggatgccataccgctgccgacagcggtgccattcaccagggctaaaccttccttgggttgcaaatcaaagaaaccagttgaaataccagctttctcaaatgcttccttagcggttaaggattctccgtctggaccagtggcctttgaattaggtcttcccgttaataagcctgcgatatatgaaaggggaaccaaatcaccgctggcagttattgttcctcttaagggcaacgaaggagaaatgttgtggttcaatagtgaagtgatggcctcaagaatttcaaaccttattccagagtaaccttgcaacaaagtgttcaccctaacaagcatagcagctcttgttgccgattggggtaatgtatggcaagtttcctttgtattaccgaaaataccggcgttaaggaatctgatcagttctgtttgcaaagcagtgccatttttagttcttctatgagaggtagcaccaaagcctgtggtaacgccataggaatctgtgcccttgttcatactttccatgacccaatctgatgaagccttaactccggctctacttgtttctgcaagttctaccttcactgaaccgccaacggtcgaaatagcagctacctgtcctatcgtcaatgtctcgccgcctagatttacgactggtcttctgtattcctcaaccatcttcttaacttcatccagatggctacctttcatctggtcagctgccagaccccaattcaaaggatctgcaagagtttttgtcgttacggccaccttggtcttttcaccaccaccgcatagcattgcttcaatttggtccattttgtatctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagatacaaaatggatttgttattgctggaaaagtcacttattgctgtatttgtggcagttattctagccacggttatttctaaattaagaggtaagaaactaaaactacctcctggtcccatccccataccaatttttggtaattggttgcaagtgggcgatgatttgaatcacagaaatttggtagactatgctaagaagttcggtgaccttttcttgcttagaatgggtcaaaggaatttggtagtggttagctcacctgatttgactaaggaggtcttattaacgcaaggcgttgagtttggctccagaactagaaatgttgtgtttgatattttcactggtaaaggtcaagatatggtttttacagtttacggtgagcactggagaaaaatgagaagaatcatgaccgtaccattctttactaacaaggttgttcaacaaaatagagaaggttgggagtttgaggcagcttccgtagtggaagacgtaaagaaaaatccagattcggccacaaagggtatagtactaagaaaaagactacaattgatgatgtacaacaatatgttcagaattatgtttgacagaagatttgaaagtgaagatgaccctttgttcctgagacttaaggctttgaatggtgaaagatcgagattggctcaaagtttcgaatataattacggtgactttattccaatcttaagaccatttttgagaggctatttgaaaatttgccaagacgtcaaggataggaggatcgctcttttcaagaagtactttgtggacgagagaaagcaaatagcttcttccaagcccacaggttcggaaggtttaaaatgtgcaattgatcatattttagaagctgaacaaaaaggtgaaattaacgaagataatgttttgtacattgtagaaaatatcaatgtggctgcaatagaaacaaccttatggtcaatagaatggggtattgctgaattggtgaatcacccagaaatacaatctaaactgagaaacgagctagataccgttttaggtccaggtgtccaagttacagaacctgatttgcataagttaccctacttgcaagctgtggttaaagaaaccttgagattgagaatggctattcctcttctagttcctcatatgaacctacatgatgctaaactggccggttatgatattccagcagaaagtaagattttagtaaatgcatggtggttggccaacaatccaaacagttggaaaaagcctgaagaattcagacctgaaagattcttcgaagaggaatctcatgttgaagccaacggaaatgacttcagatatgtaccttttggcgttggcagaagatcgtgtccaggaataatactagccttaccaatattgggtatcacaattggtaggatggttcaaaattttgagttgctaccaccacccggacaatcgaaagtcgatacttcagagaaaggaggacaattctcattgcatattttgaatcattccattatagtcatgaaacccagaaattgtccatctactccatctactccatctactccatctactaggaggagcggttcgggcaattcaaagagggttgaaccactaaagccattagttatcaaacctagagaagaggaaattgacgatggaaggaagaaagtcactatattcttcggcacccaaacaggtacagctgaaggttttgctaaggctctaggagaagaagcaaaagctagatatgaaaagacgagattcaaaattgtcgatctggatgactatgccgccgatgatgacgaatacgaagaaaaattgaagaaagaagatgtcgcatttttcttccttgccacctacggcgacggtgaaccaacagataatgccgcaaggttttacaagtggtttactgaaggtaatgacagaggagaatggctgaagaatttgaaatatggtgtgttcggccttggtaacagacagtacgagcattttaataaggtcgctaaggttgtagatgatatacttgttgaacaaggtgctcaaaggttagtgcaggtgggcttgggtgacgatgatcaatgtattgaagatgactttactgcttggagagaagccttgtggcctgaattagatactatccttagagaagaaggtgacactgctgttgctaccccctacactgcagcagtcctagaatatagagtctcaatccatgattcagaagacgccaaattcaatgatattaacatggccaacggtaacggttacaccgtttttgacgcacaacatccatacaaagctaatgttgctgttaaaagggaacttcacaccccagaaagtgacaggtcatgtatacatttggaatttgatatcgctggtagtggtttgacttacgaaacaggtgaccatgtcggagtactttgcgataatttgtcagaaactgttgatgaagctttgaggttattggatatgtcaccagatacttacttctcattgcatgcagaaaaagaagacggaactccaatatcaagctcgcttccccctccattccctccctgtaacttaagaacagccctaactagatatgcttgtttactgtcttctccaaagaaaagtgctttggttgcattggcagcccacgcatccgatcctaccgaagctgagagattaaagcatttggcttcaccagccggtaaagatgaatacagtaagtgggtagtggagagccaaagatcgcttttagaagtgatggctgagtttccaagtgctaaacctcctctgggtgtatttttcgctggtgtggccccaagattgcagcctagattttattccatatcctcatctccaaaaattgccgaaaccagaattcacgtgacatgtgctctggtctacgaaaagatgccaacaggtaggattcacaagggtgtctgttctacctggatgaaaaatgctgtaccctatgaaaaatccgaaaattgttctagtgcaccaattttcgtaagacaatctaatttcaagttaccaagcgattctaaagtacccattattatgatcggtccaggtactggtttggccccattcagaggcttcttgcaagaaagattggctttagtggagagtggagttgaattgggtccttcagttttattctttggttgtagaaacagaagaatggactttatctacgaagaagaattgcagagatttgttgaaagtggtgcattggccgaattgagtgttgcattcagcagggaaggtccaaccaaagaatacgttcaacacaagatgatggacaaggcttctgatatctggaatatgatttcccaaggtgcttatttgtatgtttgtggtgacgctaaaggaatggctagagatgttcatagatcactgcatacaatcgcacaagaacaaggtagcatggattcaacaaaagcagagggctttgtaaagaatcttcagacaagcggtagatatctgagagatgtatggtaaggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 11 - DNA, 35 bp, artificial
  primer
cactaaagggcggccgcatggaccaaattgaagca

SEQ ID NO: 12 - DNA, 38 bp, artificial
  primer
aattaagagctcagatctttagcagattggaataggtg

SEQ ID NO: 13 - DNA, 40 bp, artificial
  primer
atttccgaagaagacctcgagatggatttgttattgctgg

SEQ ID NO: 14 - DNA, 55 bp, artificial
  primer
agtagatggagtagatggagtagatggagtagatggacaatttctgggtttcatg

SEQ ID NO: 15 - DNA, 52 bp, artificial
  primer
ccatctactccatctactccatctactccatctactaggaggagcggttcgg

SEQ ID NO: 16 - DNA, 44 bp, artificial
  primer
atcttagctagccgcggtaccttaccatacatctctcagatatc

SEQ ID NO: 17 - DNA, 34 bp, artificial
  primer
ccggatcctcatggcatccgtcgaagagttcagg

SEQ ID NO: 18 - DNA, 38 bp, artificial
  primer
cgctcgagttttagttagtaactgtgggaacgctatgc

SEQ ID NO: 19 - DNA, 40 bp, artificial
  primer
gcgaattcttatgacgacacaagatgtgatagtcaatgat

SEQ ID NO: 20 - DNA, 41 bp, artificial
  primer
gcactagtatcctagttcattaatccatttgctagtcttgc

SEQ ID NO: 21 - DNA, 40 bp, artificial; identical to SEQ ID NO: 13
  primer

SEQ ID NO: 22 - DNA, 55 bp, artificial; identical to SEQ ID NO: 14
  primer

SEQ ID NO: 23 - DNA, 52 bp, artificial; identical to SEQ ID NO: 15
  primer

SEQ ID NO: 24 - DNA, 44 bp, artificial; identical to SEQ ID NO: 16
  primer

SEQ ID NO: 25 - DNA, 40 bp, artificial; identical to SEQ ID NO: 13
  primer

SEQ ID NO: 26 - DNA, 44 bp, artificial; identical to SEQ ID NO: 16
  primer

SEQ ID NO: 27 - DNA, 41 bp, artificial
  primer
gcgagctcagtttatcattatcaatactcgccatttcaaag

SEQ ID NO: 28 - DNA, 46 bp, artificial
  primer
cgtctagaatccgtcgaaactaagttctggtgttttaaaactaaaa

SEQ ID NO: 29 - DNA, 44 bp, artificial
  primer
gcgagctcatagcttcaaaatgtttctactccttttttactctt

SEQ ID NO: 30 - DNA, 45 bp, artificial
  primer
cgtctagaaaacttagattagattgctatgctttctttctaatga

SEQ ID NO: 31 - DNA, 50 bp, artificial
  primer
ttgcgtattgggcgctcttccgagctcagtttatcattatcaatactcgc

SEQ ID NO: 32 - DNA, 35 bp, artificial
  primer
atggatcctctagaatccgtcgaaactaagttctg

SEQ ID NO: 33 - DNA, 41 bp, artificial
  primer
atgaattctctagaaaacttagattagattgctatgctttc

SEQ ID NO: 34 - DNA, 44 bp, artificial
  primer
tgataatgataaactgagctcggaagagcgcccaatacgcaaac

SEQ ID NO: 35 - DNA, 41 bp, artificial; identical to SEQ ID NO: 33
  primer

SEQ ID NO: 36 - DNA, 35 bp, artificial; identical to SEQ ID NO: 32
  primer

SEQ ID NO: 37 - DNA, 35 bp, artificial
  primer
atgaattctctagaatccgtcgaaactaagttctg

SEQ ID NO: 38 - DNA, 48 bp, artificial
  primer
atggatcctctagaaaacttagattagattgctatgctttctttctaa

SEQ ID NO: 39 - DNA, 50 bp, artificial
  primer
ttccagcaataacaaatccattttgtatctagaaaacttagattagattg

SEQ ID NO: 40 - DNA, 50 bp, artificial
  primer
cattgcttcaatttggtccattttgtatctagaatccgtcgaaactaagt